(19) United States
(12) Patent Application Publication
(10) Pub. No.: US 2024/0254553 A1
(43) Pub. Date: Aug. 1, 2024

(54) Multiple Feature Integration With Next-Generation Three-Dimensional in Situ Sequencing

(71) Applicant: The Board of Trustees of the Leland Stanford Junior University, Stanford, CA (US)

(72) Inventors: Karl A. Deisseroth, Stanford, CA (US); Ethan B. Richman, Palo Alto, CA (US)

(21) Appl. No.: 18/561,262

(22) PCT Filed: May 20, 2022
(86) PCT No.: PCT/US2022/030363

§ 371 Date: Nov. 15, 2023

Related U.S. Application Data
(60) Provisional application No. 63/191,457, filed on May 21, 2021.

(51) Int. Cl. C12Q 1/6874; C12N 15/10; C12Q 1/6841

(52) U.S. Cl. CPC C12Q 1/6874; C12N 15/1065; C12Q 1/6841

(57) ABSTRACT

Provided herein are devices, methods, and systems for multiple feature integration with next-generation three-dimensional in situ sequencing of nucleic acids in cells in intact tissue. Biological samples contain many distinct types of molecular, cellular, anatomical, and experimental features. The disclosed methods allow simultaneous interrogation of multiple distinct features of a biological sample, including RNA features, anatomical features, exogenous barcodes, or other arbitrary experimental features such as in vivo measurements, which can be combined into single experimental readouts with next-generation in situ sequencing.

### BACKGROUND OF THE INVENTION

Biological samples contain complex and heterogenous genetic information spanning the length scales of individual cells and whole tissues. Spatial patterns of nucleic acids within a cell may reveal properties and abnormalities of cellular function; cumulative distributions of RNA expression may define a cell type or function; and systematic variation in the locations of cell types within a tissue may define tissue function. The combination of anatomical connectivity information encoded in nucleic acids and tissue-wide cell type distributions may span many sections of tissue. Techniques for in situ nucleic acid sequencing must therefore be able to bridge resolutions as small as individual molecules and as large as entire brains. Efficiently collecting and recording this information across orders-of-magnitude differences in lengths requires novel inventions to enhance the robustness, rapidity, automated-, and high throughput-nature of in situ sequencing techniques.

### SUMMARY OF THE INVENTION

Provided herein are devices, methods, and systems for multiple feature integration with next-generation three-dimensional in situ sequencing of nucleic acids in cells in intact tissue. Biological samples contain many distinct types of molecular, cellular, anatomical, and experimental features. The disclosed methods allow simultaneous interrogation of multiple distinct features of a biological sample, including RNA features, anatomical features, exogenous barcodes, or other arbitrary experimental features such as in vivo measurements, which can be combined into single experimental readouts with next-generation in situ sequencing.

In one aspect, a method of in situ sequencing of a target nucleic acid in a cell in an intact tissue in combination with cell barcoding is provided, the method comprising: introducing into the cell in the intact tissue a viral vector comprising a promoter operably linked to a sequence encoding a messenger RNA (mRNA) transcript comprising a 3′-untranslated region (3′-UTR) comprising a cell barcode and a poly-adenylation site, wherein the cell barcode is adjacent to the poly-adenylation site; measuring morphological or functional characteristics of the cell in the intact tissue; sequencing the barcode of the mRNA transcript; and performing in situ gene sequencing of the target nucleic acid in the cell in the intact tissue, wherein the cell barcode is used for assignment of in situ sequencing data to the measured morphological or functional characteristics of the cell.

In certain embodiments, measuring morphological or functional characteristics comprises performing gene expression profiling, microscopy (e.g., confocal microscopy, atomic force microscopy, super-resolution microscopy, light-sheet microscopy, two-photon microscopy, or fluorescence microscopy), calcium imaging, electrophysiology measurements (e.g., patch clamping, electroencephalography (EEG), and magnetoencephalography (MEG)), functional neuroimaging (e.g., functional magnetic resonance imaging (fMRI), positron emission tomography (PET), functional near-infrared spectroscopy (fNIRS), single-photon emission computed tomography (SPECT), or functional ultrasound imaging (fUS). functional magnetic resonance imaging (fMRI)), migration assays, axonal growth and pathfinding assays, phagocytosis assays, enzymatic assays, cell receptor assays, ion channel assays, signal transduction assays, or cell secretion assays, or any combination thereof.

In certain embodiments, the viral vector is introduced into the cell in vivo, ex vivo, or in vitro prior to said measuring the morphological or functional characteristics of the cell.

In certain embodiments, the morphological or functional characteristics are measured in tissue of a live subject followed by removal of the intact tissue from the subject prior to said performing in situ gene sequencing.

In certain embodiments, the subject is a nonhuman animal.

In certain embodiments, the method further comprises removing the intact tissue from the subject prior to said measuring morphological or functional characteristics of the cell in the intact tissue and said performing in situ gene sequencing.

In certain embodiments, the intact tissue is a biopsy or surgical specimen.

In certain embodiments, the viral vector is an adeno-associated virus (rAAV) vector.

In certain embodiments, the mRNA transcript further comprises a coding sequence encoding a protein. In some embodiments, the protein is a fluorescent protein or a bioluminescent protein. In some embodiments, the method further comprises imaging the fluorescent protein or the bioluminescent protein, wherein a location of the cell expressing the fluorescent protein or the bioluminescent protein is determined from the imaging. In certain embodiments, the method further comprises mapping the location of the cell expressing the fluorescent protein or the bioluminescent protein onto a reference image of the intact tissue. In some embodiments, the method further comprises mapping the in situ sequencing data onto the reference image of the intact tissue.

In certain embodiments, the cell is a neuron. In some embodiments, the neuron is a projection neuron. In some embodiments, the viral vector is introduced into a projection of the projection neuron, wherein retrograde transport of the viral vector delivers the viral vector to the cell body of the projection neuron. For example, the viral vector may be introduced into a projection of the projection neuron by stereotactic injection.

In certain embodiments, the method further comprises optogenetically modifying one or more cells in the intact tissue.

In certain embodiments, the intact tissue is brain tissue.

In certain embodiments, the method further comprises mapping functional neuroimaging data onto the reference image of the intact tissue.

In certain embodiments, the method further comprises fixing and permeabilizing the intact tissue.

In certain embodiments, sequencing the barcode of the mRNA transcript comprises performing single-cell 3′-RNA sequencing of the mRNA transcript.

In certain embodiments, performing in situ gene sequencing comprises: (a) contacting the fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon; (f) reiterating step (e); and (g) imaging the one or more hydrogel-embedded amplicons to determine in situ a gene sequence of the target nucleic acid in the cell in the intact tissue.

In certain embodiments, the target nucleic acid is the mRNA transcript comprising the 3′-untranslated region (3′-UTR) comprising the cell barcode and the poly-adenylation site that was introduced in the cell with a viral vector, wherein said imaging is used to determine the sequence of the cell barcode. In some embodiments, the length of the cell barcode sequence is sufficient to allow at least one pair of oligonucleotide primers to bind to the cell barcode sequence, wherein the first complementarity region of the first oligonucleotide is complementary to a first portion of the barcode sequence, wherein the second complementary region of the second oligonucleotide is complementary to a second portion of the barcode sequence, and wherein the first portion of the barcode sequence is adjacent to the second portion of the barcode sequence. In some embodiments, the length of the cell barcode sequence is sufficient to allow at least two pairs of oligonucleotide primers to bind to the cell barcode sequence. In some embodiments, the length of the cell barcode sequence is sufficient to allow at least four pairs of oligonucleotide primers to bind to the cell barcode sequence. In some embodiments, the length of the cellular barcode sequence in the mRNA transcript is sufficient for 1 to 5 pairs of oligonucleotide primers to bind to the cellular barcode sequence, including any number within this range such as 1, 2, 3, 4, or 5 pairs of oligonucleotide primers, wherein the oligonucleotide primers have complementarity regions that are complementary to a portion of the cellular barcode sequence. In some embodiments, the cell barcode sequence has a length of at least 30 nucleotides, at least 40 nucleotides, at least 50 nucleotides, or at least 60 nucleotides.

In certain embodiments, the method further comprises contacting the fixed and permeabilized intact tissue with a gel adaptor oligonucleotide that binds to the first oligonucleotide, wherein the gel adaptor oligonucleotide comprises a nucleotide modification at the 5′ end that links the gel adapter to the hydrogel during gelation. In some embodiments, the modification comprises an acrydite group. In some embodiments, the first oligonucleotide further comprises a common binding site for the gel adaptor oligonucleotide. In some embodiments, the common binding site for the gel adaptor oligonucleotide is adjacent to the first complementarity region of the first oligonucleotide.

In certain embodiments, the method further comprises barcoding a cell by contacting the cell with: a first probe comprising a 5′-amine modification or a 5′-biotin modification, a common gel adaptor complementary sequence that hybridizes with the gel adaptor oligonucleotide, and a unique barcode sequence; and a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe. In some embodiments, the second probe is a padlock probe. In some embodiments, a plurality of first probes and second probes are used to barcode a plurality of cells in the intact tissue, wherein each first probe has a different unique barcode sequence.

In certain embodiments, sequencing is performed with sequential or combinatorial encoding.

In certain embodiments, the method further comprises preincubating the tissue sample with the polymerase for a sufficient time to allow uniform diffusion of the polymerase throughout the tissue before performing the rolling circle amplification.

In certain embodiments, the signal is a fluorescent signal.

In certain embodiments, the imaging is performed in presence of an anti-fade buffer comprising an antioxidant.

In certain embodiments, the method further comprises removing the signal after imaging by contacting the hydrogel with formamide.

In certain embodiments, the fourth oligonucleotide is covalently linked to a fluorophore by a disulfide bond. In some embodiments, the method further comprises contacting the hydrogel with a reducing agent after said imaging, wherein reduction of the disulfide bond results in cleavage of the fluorophore from the fourth oligonucleotide.

In certain embodiments, the set of primers are denatured by heating before contacting the sample.

In certain embodiments, the cell is present in a population of cells. In some embodiments, the population of cells comprises a plurality of cell types.

In certain embodiments, the contacting the fixed and permeabilized intact tissue comprises hybridizing the primers to the same target nucleic acid.

In certain embodiments, the target nucleic acid is RNA or DNA. In some embodiments, the RNA is mRNA.

In certain embodiments, the second oligonucleotide comprises a padlock probe.

In certain embodiments, the first complementarity region of the first oligonucleotide has a length of 19-25 nucleotides.

In certain embodiments, the second complementarity region of the first oligonucleotide has a length of 6 nucleotides.

In certain embodiments, the third complementarity region of the first oligonucleotide has a length of 6 nucleotides.

In certain embodiments, the first complementarity region of the second oligonucleotide has a length of 6 nucleotides.

In certain embodiments, the second complementarity region of the second oligonucleotide has a length of 19-25 nucleotides.

In certain embodiments, the third complementarity region of the second oligonucleotide has a length of 6 nucleotides.

In certain embodiments, the first complementarity region of the second oligonucleotide comprises the 5′ end of the second oligonucleotide.

In certain embodiments, the third complementarity region of the second oligonucleotide comprises the 3′ end of the second oligonucleotide.

In certain embodiments, the first complementarity region of the second oligonucleotide is adjacent to the third complementarity region of the second oligonucleotide.

In certain embodiments, the barcode sequence of the second oligonucleotide provides barcoding information for identification of the target nucleic acid.

In certain embodiments, the contacting the fixed and permeabilized intact tissue comprises hybridizing a plurality of oligonucleotide primers having specificity for different target nucleic acids.

In certain embodiments, the second oligonucleotide is provided as a closed nucleic acid circle, and the step of adding ligase is omitted.

In certain embodiments, the melting temperature (Tₘ) of oligonucleotides is selected to minimize ligation in solution.

In certain embodiments, adding ligase comprises adding a DNA ligase.

In certain embodiments, the nucleic acid molecule comprises an amine-modified nucleotide.

In some embodiments, the amine-modified nucleotide comprises an acrylic acid N-hydroxysuccinimide moiety modification.

In certain embodiments, the embedding comprises copolymerizing the one or more amplicons with acrylamide.

In certain embodiments, the embedding comprises clearing the one or more hydrogel-embedded amplicons wherein the target nucleic acid is substantially retained in the one or more hydrogel-embedded amplicons.

In certain embodiments, the clearing comprises substantially removing a plurality of cellular components from the one or more hydrogel-embedded amplicons. In some embodiments, the clearing comprises substantially removing lipids or proteins, or a combination thereof from the one or more hydrogel-embedded amplicons.

In certain embodiments, contacting the one or more hydrogel-embedded amplicons comprises eliminating error accumulation as sequencing proceeds.

In certain embodiments, the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

In certain embodiments, the intact tissue is a thin slice. In some embodiments, the intact tissue has a thickness of 5-20 μm. In some embodiments, the contacting the one or more hydrogel-embedded amplicons occurs four times or more.

In certain embodiments, the intact tissue is a thick slice. In some embodiments, the intact tissue has a thickness of 50-200 μm. In some embodiments, the contacting the one or more hydrogel-embedded amplicons occurs six times or more.

In another aspect, a method of screening a candidate agent to determine whether the candidate agent modulates gene expression of a nucleic acid in a cell in an intact tissue is provided, the method comprising performing the method described herein to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain embodiments, the detecting comprises performing flow cytometry (e.g., mass cytometry or fluorescence-activated flow cytometry); sequencing; probe binding and electrochemical detection; pH alteration; catalysis induced by enzymes bound to DNA tags; quantum entanglement; Raman spectroscopy; terahertz wave technology; and/or scanning electron microscopy. In certain embodiments, the detecting comprises performing microscopy, scanning mass spectrometry, or other imaging techniques.

In certain embodiments, the detecting comprises detecting a signal. In some embodiments, the signal is a fluorescent signal.

In another aspect, a system is provided, the system comprising: a fluidics device, and a processor unit configured to perform a method described herein.

In certain embodiments, the system further comprises an imaging chamber.

In certain embodiments, the system further comprises a pump.

### BRIEF DESCRIPTION OF THE DRAWINGS

FIG. 1. Integrating multiple features across scales in neural circuits. From left to right: measurements of a live animal's behavior during an operant task; neural activity as read out by two-photon imaging of calcium activity sensed by GCaMP; mesoscale projection connectivity information via retro-trafficking of area-specific barcodes delivered by AAVretro; in situ sequencing of endogenous gene and barcode expression for the identification of functional, molecular, and anatomical cell types. Features across scales are integrated through the combined usage of volumetric alignments, barcoded information, and gene expression measurements.

FIGS. 2A-2C. Combining anatomical projection mapping, activity imaging during behavior, and in situ sequencing in a single animal. (FIG. 2A) Schematic of the experimental approach. Animals are first injected with AAVretro viruses containing distinct barcoded sequences to label different projection targets of a given region of study. The region of study is additionally labeled with GCaMP for fluorescent 2-photon calcium imaging. Activity and reference volumes from the live animal are collected during and after animal behavior once viral constructs have sufficiently expressed. Following the completion of the in vivo experimental stage, the animal is sacrificed and the in vivo imaged region is collected and processed via STARmap2 in situ sequencing. (FIG. 2B) Calcium imaging data collected from thousands of cells in the mouse orbital frontal cortex during behavior. (FIG. 2C) Data from the same mouse, showing the volumetric alignment process. From left to right: extracted sources from the in vivo activity volume; alignment of the in vivo activity volume to an in vivo reference volume; alignment of the in vivo reference volume to an ex vivo reference volume (collected as part of the STARmap2 sequencing). Applying the resulting series of transformations maps segmented cells from the STARmap2 dataset to the activity imaging image volume space (right).

FIG. 3. Detail on the in vivo (2-photon imaging) to ex vivo (STARmap2 data) registration pipeline. In vivo and ex vivo volumes are manually roughly aligned via rotation in the XY plane and pixel scaling according to the two imaging system configurations. An affine transformation is followed by a warping transformation, resulting in the middle warped volume, which is now aligned across in vivo and ex vivo image volume spaces. Evaluation of the local volume normalized cross correlation at a length scale greater than the granularity of the warping procedure yields an alignment quality score that can be used to exclude volume with insufficiently accurate alignment.

FIGS. 4A-4F. Using barcoded viruses to connect mesoscale neuron projection data to molecule-scale cell type information in the mouse orbital frontal cortex. (FIG. 4A) Four different barcoded AAVretro preparations are injected into projection targets of the orbital frontal cortex: contralateral OFC (contra OFC), dorsal striatum (striatum), medial-dorsal thalamus (MD Thalamus/thalamus), and the ventral tegmental area (VTA)/substantia nigra pars compacta (SNc). After injections, viral constructs are allowed to traffic and express for several weeks. (FIG. 4B) Thin section STARmap2 of the OFC of an animal injected with the barcoded AAVretro constructs. 1, a contralateral OFC projecting cell; 2, a thalamic projecting cell; 3, a cell with collateralizing projections to both MD Thalamus and VTA/SNc; 4, a cell projecting to dorsal striatum; all cell projection anatomy are identified by quantifying the barcode expression with STARmap2 sequencing. (FIG. 4C) Cell types in the OFC identified by sequencing of 48 cell-type marker genes in an example 150 μm thick tissue section from an animal that received the four barcoded AAVretro injections. (FIG. 4D) Quantification of marker gene expression in identified cell-type clusters in the STARmap2 data from 6 animals. (FIG. 4E) Average normalized presence of barcodes for different projection targets (x axis) in the various cell types segmented in the STARmap2 data, from the same 6 animals as (FIG. 4D). (FIG. 4F) Distribution of observation frequency for different collateralization patterns observed in OFC cells as deteceted by STARmap2, from the same animals as in D and E. White squares in the bottom indicate the presence of a target; multiple white squares indicate a projection type that projects to multiple targets.

FIG. 5. Detail on the preparation of matching tissue sections from mouse brains that have been previously imaged. From left to right: a mouse brain dissected out from the animal such that the imaging canula remains implanted in the brain and the brain attached to the headbar used during behavior and imaging; the brain tissue is embedded on the vibratome cutting platform by attaching the headbar to a headbar holder (such that it maintains the same position relative to the perpendicular platform as it had during imaging), gluing the bottom of the brain to the vibratome cutting platform, and then lowering the platform away from the headbar to separate the brain from the headbar and imaging canula; thick tissue sections collected in series until the hole remaining from the imaging canula disappears (sections marked with arrows), resulting in the bottom two sections containing tissue volumes that were imaged in vivo.

FIG. 6. Assessment of alignment procedures with a ground truth native fluorescence control. Transgenic mice expressing EYFP protein in SST+ cells were imaged for both an EYFP and RFP (alignment channel) reference volumes. Following sacrifice and STARmap2 sample preparation, the STARmap2 gel was imaged for RFP and residual EYFP signals (ex vivo). The RFP reference channels (in vivo) were aligned using the computational registration methods to the ex vivo RFP signals in the STARmap2 gel. Distance between in vivo EYFP cell centers and ex vivo EYFP cell centers in the STARmap2 gel were quantified; the resulting average distance was measured to 1.78 microns, or less than the pixel size of the in vivo volume.

FIGS. 7A-7C. The STARpatch method: combining electrophysiology, cell-specific barcoding, cell-filling biocytin labeling for volumetric cell morphology, and STARmap2 volumetric combinatorial sequencing of gene expression information. (FIG. 7A) The experimental approach, consisting of whole-cell patch-clamp recording of neurons with a patch pipette filled with a cell-specific barcoding oligo complex and biocytin (for cell morphology) in addition to the internal solution. Left, example electrophysiological data collected from a patched, barcoded, and biocytin-filled cell. (FIG. 7B) Following STARmap2 preparation of the section, including an oligo-conjugated streptavidin for biocytin detection and linkage to the hydrogel, the oligo sequence is detected by hybridization with a fluorophore containing oligo during the morphology round of the STARmap2 sequencing, resulting in a high-SNR volumetric reconstruction of the patched neuron and its 3D morphology. (FIG. 7C) Cell-specific barcode signal, introduced to the cell during patching, detected during STARmap2 sequencing. Arrowhead indicates location of the detected cell (by concentration of amplicons with the cell-specific barcode information).

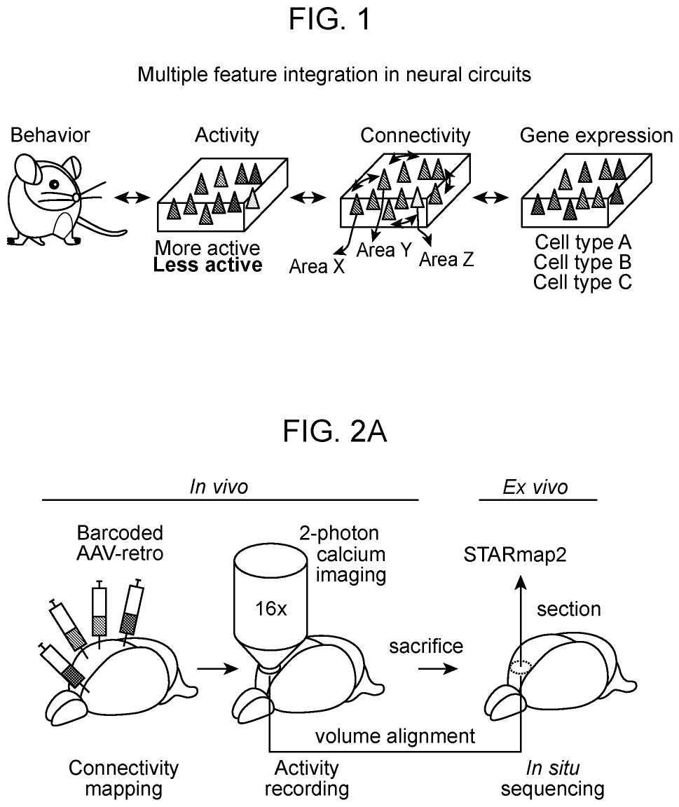

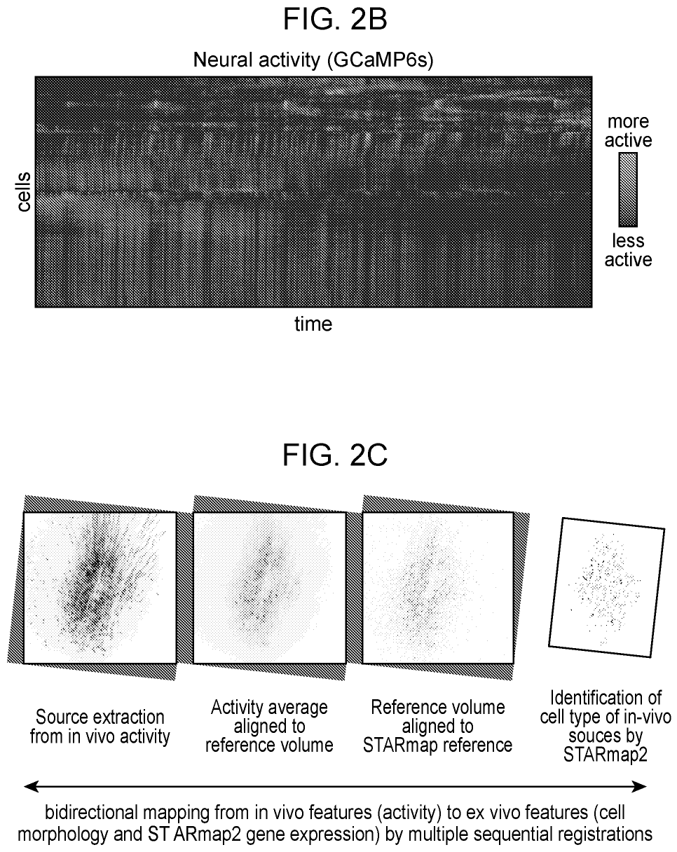

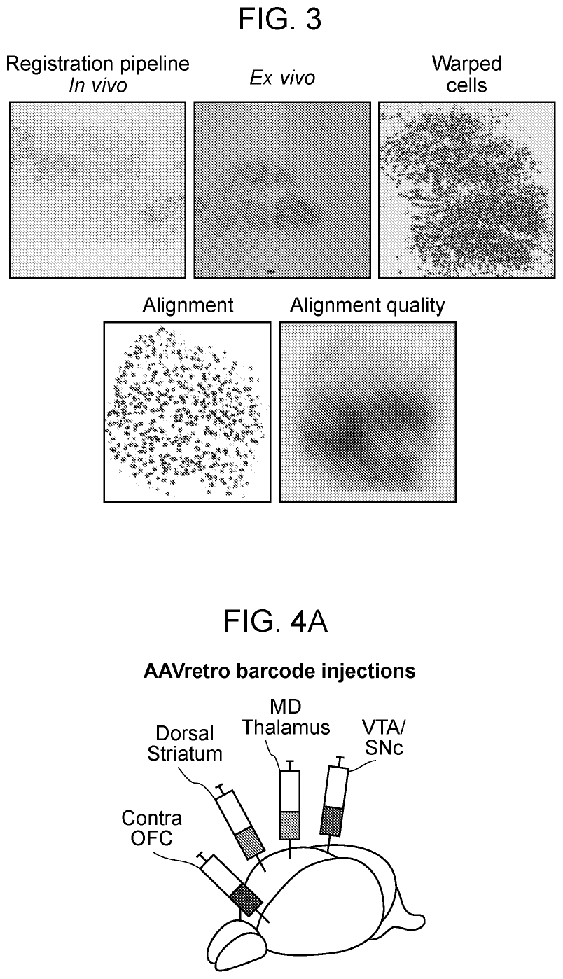

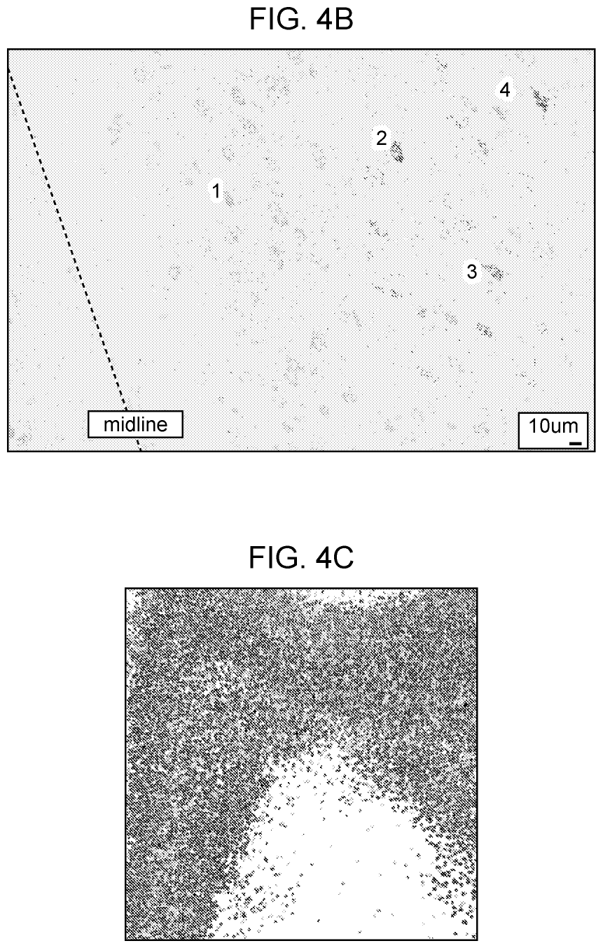

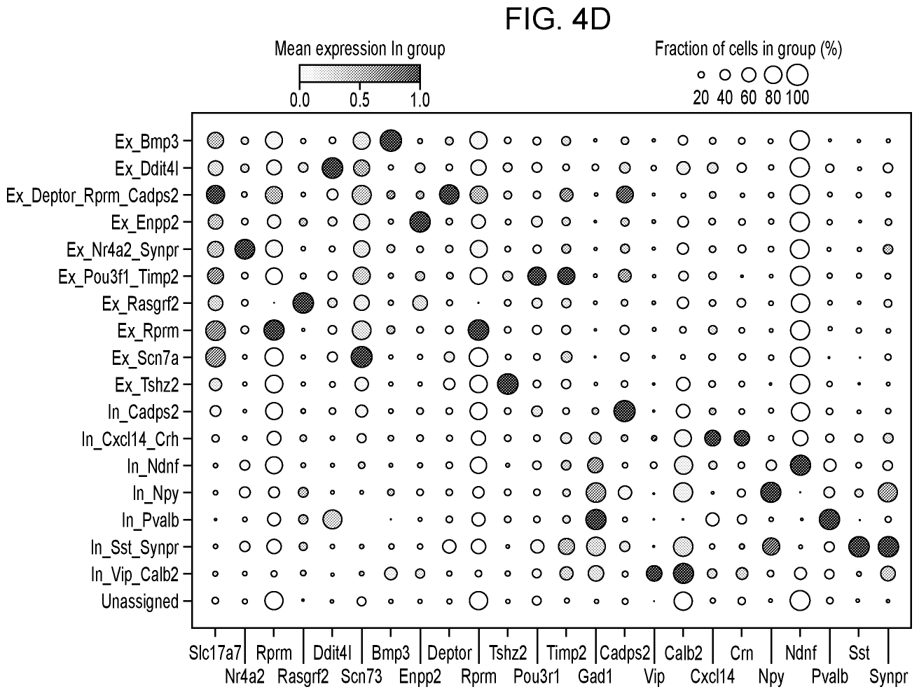

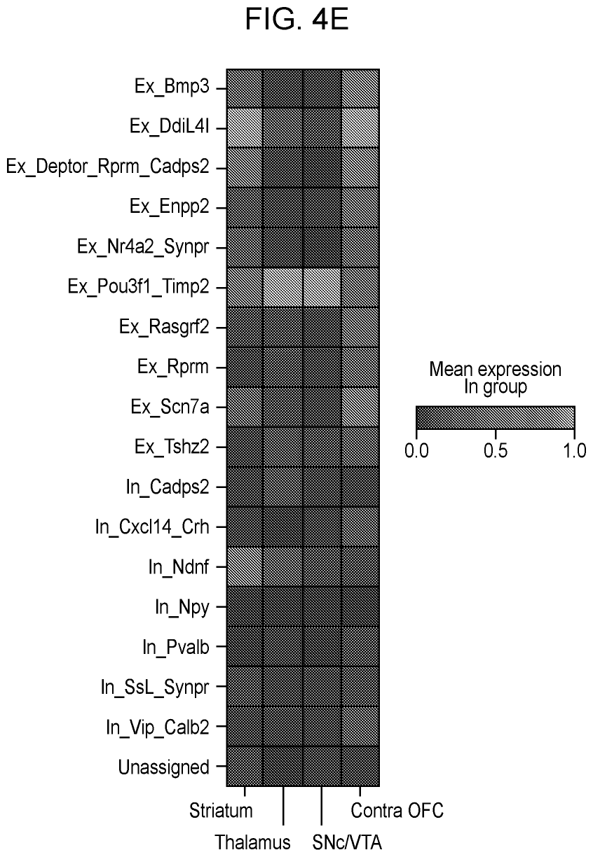

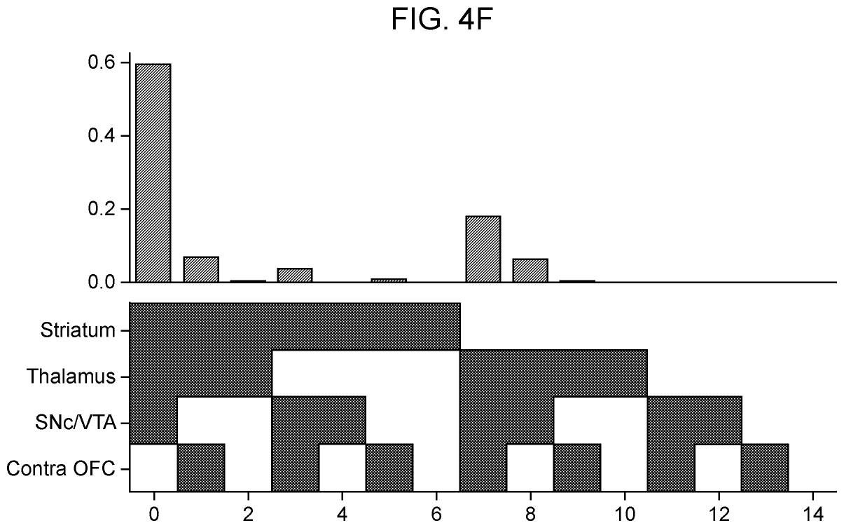

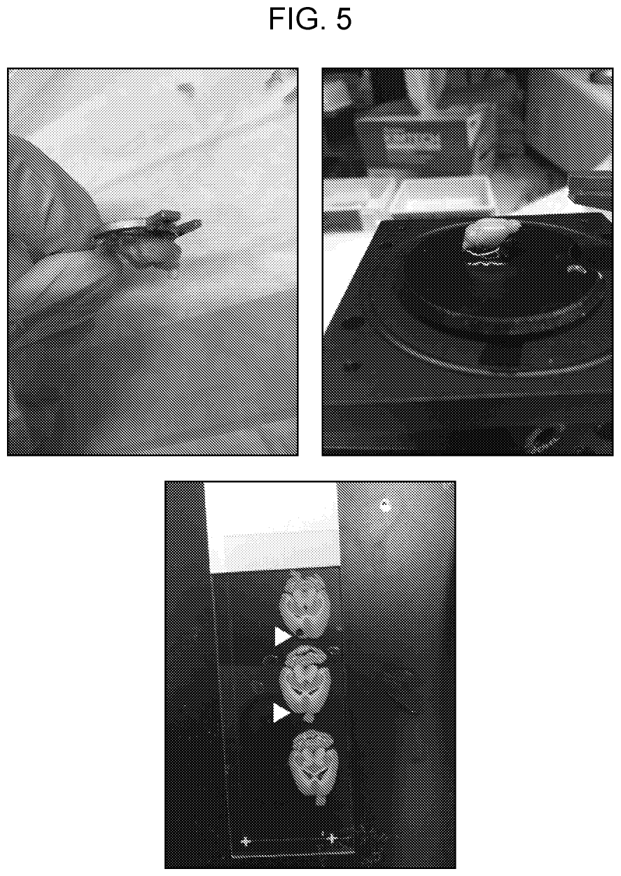

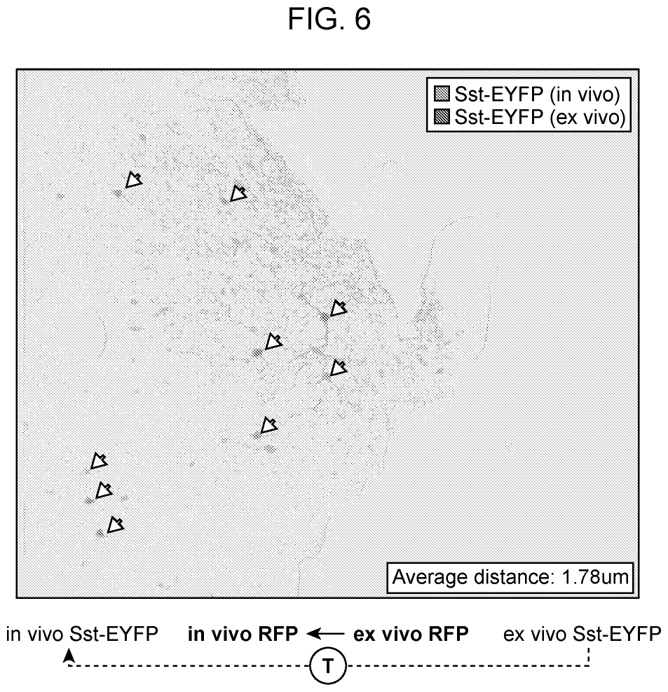

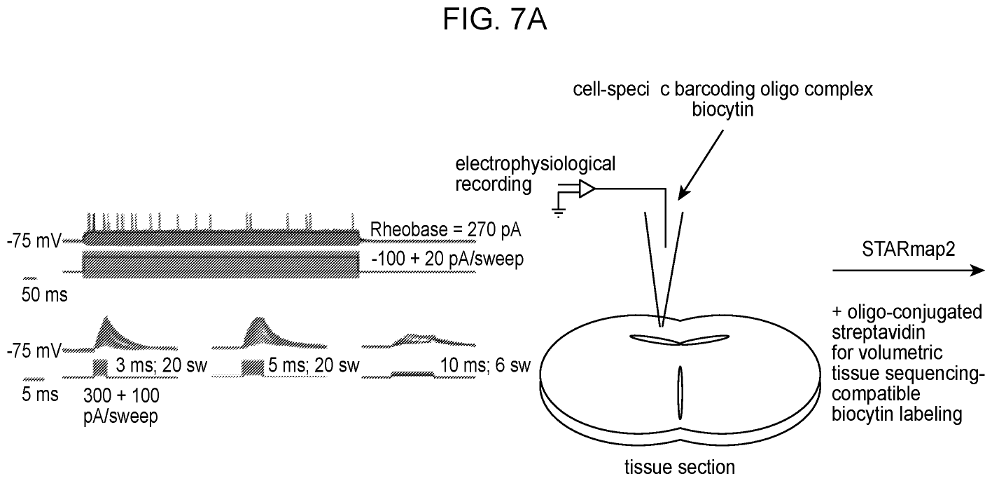

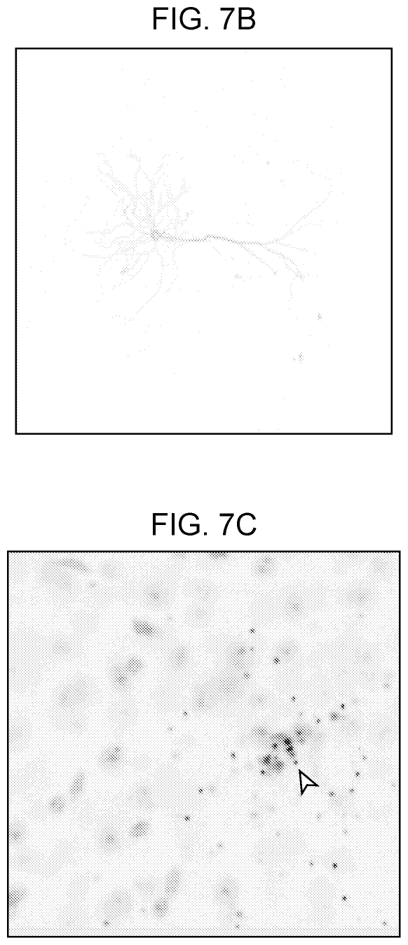

### DETAILED DESCRIPTION OF THE INVENTION

Provided herein are devices, methods, and systems for multiple feature integration with next-generation three-dimensional in situ sequencing of nucleic acids in cells in intact tissue. Biological samples contain many distinct types of molecular, cellular, anatomical, and experimental features. The disclosed methods allow simultaneous interrogation of multiple distinct features of a biological sample, including RNA features, anatomical features, exogenous barcodes, or other arbitrary experimental features such as in vivo measurements, which can be combined into single experimental readouts with next-generation in situ sequencing.

Before the present devices, methods, and systems are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an” and “the” include plural referents unless the context clearly dictates otherwise. Thus, for example, reference to “a cell” includes a plurality of such cells and reference to “the peptide” includes reference to one or more peptides and equivalents thereof, e.g. oligopeptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

As used herein, the term “target nucleic acid” is any polynucleotide nucleic acid molecule (e.g., DNA molecule; RNA molecule, modified nucleic acid, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA; etc.). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, i.e. have a differing abundance, within a cell population, wherein the methods of the invention allow profiling and comparison of the expression levels of nucleic acids, including without limitation RNA transcripts, in individual cells. A target nucleic acid can also be a DNA molecule, e.g. a denatured genomic, viral, plasmid, etc. For example, the methods can be used to detect copy number variants, e.g. in a cancer cell population in which a target nucleic acid is present at different abundance in the genome of cells in the population; a virus-infected cells to determine the virus load and kinetics, and the like.

The terms “oligonucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The backbone of the polynucleotide can include sugars and phosphate groups (as may typically be found in RNA or DNA), or modified or substituted sugar or phosphate groups. Alternatively, the backbone of the polynucleotide can include a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may include one or more L-nucleosides. A polynucleotide may include modified nucleotides, such as methylated nucleotides and nucleotide analogs, uracil, other sugars, and linking groups such as fluororibose and thioate, and nucleotide branches. The sequence of nucleotides may be interrupted by non-nucleotide components. A polynucleotide may be modified to include N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein. A polynucleotide used in amplification is generally single-stranded for maximum efficiency in amplification, but may alternatively be double-stranded. If double-stranded, the polynucleotide can first be treated to separate its strands before being used to prepare extension products. This denaturation step is typically affected by heat, but may alternatively be carried out using alkali, followed by neutralization.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “individual”, “subject”, “host”, and “patient”, are used interchangeably herein and refer to invertebrates and vertebrates including, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M.O. in Atlas of Protein Sequence and Structure M.O. Dayhoff ed., 5 Suppl. 3:353 358, National biomedical Research Foundation, Washington, DC, which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, adenoviruses, lentiviruses, alphaviruses, pox viruses, and vaccinia viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

### Methods

The disclosed methods allow simultaneous interrogation of multiple distinct features of a biological sample, including RNA features, anatomical features, exogenous barcodes, and experimental data such as in vivo or in vitro measurements, which can be combined with next-generation in situ sequencing. In one aspect, a method of in situ sequencing of a target nucleic acid in a cell in an intact tissue in combination with cell barcoding is provided, the method comprising: introducing into the cell in the intact tissue a viral vector comprising a promoter operably linked to a sequence encoding a messenger RNA (mRNA) transcript comprising a 3′-untranslated region (3′-UTR) comprising a cell barcode and a poly-adenylation site, wherein the cell barcode is adjacent to the poly-adenylation site; measuring morphological or functional characteristics of the cell in the intact tissue; sequencing the barcode of the mRNA transcript; and performing in situ gene sequencing of the target nucleic acid in the cell in the intact tissue, wherein the cell barcode is used for assignment of in situ sequencing data to the measured morphological or functional characteristics of the cell. The viral vector may be introduced into the cell in vivo, ex vivo, or in vitro prior to measuring the morphological or functional characteristics of the cell. In some cases, the morphological or functional characteristics are measured in a live subject in vivo followed by removing tissue (e.g., biopsy, surgical specimen) or an organ from the subject prior to performing in situ gene sequencing.

In certain embodiments, the mRNA transcript further comprises a coding sequence encoding a protein. In some embodiments, the protein is a fluorescent protein or a bioluminescent protein, wherein imaging of the fluorescent protein or the bioluminescent protein can be used to determine a location of a cell expressing the mRNA encoding the fluorescent protein or the bioluminescent protein. In certain embodiments, the method further comprises mapping the location of the cell expressing the mRNA encoding the fluorescent protein or the bioluminescent protein onto a reference image of the intact tissue. In some embodiments, the method further comprises mapping in situ sequencing data onto the reference image of the intact tissue.

Exemplary fluorescent proteins include, without limitation, green fluorescent protein, superfolder green fluorescent protein, enhanced green fluorescent protein, Dronpa (a photoswitchable green fluorescent protein), yellow-green fluorescent protein, yellow fluorescent protein, red fluorescent protein, orange fluorescent protein, blue fluorescent protein, cyan fluorescent protein, violet fluorescent protein, mApple, mNectarine, mNeptune, mCherry, mStrawberry, mPlum, mRaspberry, mCrimson3, mCarmine, mCardinal, mScarlet, mRuby2, FusionRed, mNeonGreen, TagRFP675, and mRFP1. The fluorescent signal of fluorescent proteins can be detected, for example, using fluorescence microscopy or fluorescence confocal laser scanning microscopy.

Exemplary bioluminescent proteins include, without limitation, aequorins and luciferases, such as, but not limited to, firefly luciferase, Renilla luciferase, Elateroidea luciferase, Metridia luciferase, Vibrio luciferase, dinoflagellate luciferase, and nano-lantern luciferase. The luminescent signal of bioluminescent proteins can be detected, for example, using luminescence microscopy, luminescence digital imaging microscopy, time-gated luminescence microscopy, or a luminometer.

In some embodiments, the subject methods are used to integrate in situ gene sequencing data with experimental measurements made by one or more techniques. For example measuring morphological or functional characteristics may comprise performing gene expression profiling, microscopy (e.g., confocal microscopy, atomic force microscopy, super-resolution microscopy, light-sheet microscopy, two-photon microscopy, or fluorescence microscopy), calcium imaging, electrophysiology measurements (e.g., patch clamping, electroencephalography (EEG), and magnetoencephalography (MEG)), functional neuroimaging (e.g., functional magnetic resonance imaging (fMRI), positron emission tomography (PET), functional near-infrared spectroscopy (fNIRS), single-photon emission computed tomography (SPECT), or functional ultrasound imaging (fUS). functional magnetic resonance imaging (fMRI)), migration assays, axonal growth and pathfinding assays, phagocytosis assays, enzymatic assays, cell receptor assays, ion channel assays, signal transduction assays, or cell secretion assays, or any combination thereof. In some embodiments, in situ gene sequencing data is combined with one or more, two or more, three or more, four or more, or five or more other types of experimental measurements, wherein cell barcoding is used to match the experimental data obtained by these measurements with the in situ sequencing data for an individual cell in the tissue.

In some embodiments, neurons in brain tissue are barcoded, as described herein, and electrophysiology measurements are made on the barcoded neurons followed by in situ sequencing of target nucleic acids in the brain tissue. Electrophysiology techniques can be used to measure electrical properties of individual barcoded neurons in the brain, for example, to monitor voltage or current changes of neurons. Exemplary electrophysiology techniques that can be used in the practice of the subject methods include, without limitation, electroencephalography (EEG), magnetoencephalography (MEG), and patch-clamping. These electrophysiology techniques are useful for identifying the specific types of neurons involved in neural networks and measuring neuron-specific changes in activity associated with brain responses. In some cases, electrophysiology measurements are made on barcoded neurons in the brain of a live subject followed by removal of a tissue specimen from the brain region where neurons were barcoded and then performing in situ sequencing of target nucleic acids in cells of the tissue specimen.

In some embodiments, EEG is used to record neuronal electrical activity in the brain. EEG can be performed noninvasively with electrodes placed on the scalp. EEG measurements have the advantage of having high temporal resolution and can detect changes in electrical activity in the brain on a millisecond time scale. For a description of EEG and methods of using EEG for recording electrical activity in the brain, see, e.g., Niedermeyer et al. (2004) Electroencephalography: Basic Principles, Clinical Applications, and Related Fields. Lippincott Williams & Wilkins; Jackson et al. (2014) Psychophysiology 51(11): 1061-71, Khanna et al. (2015) Neurosci Biobehav Rev. 49:105-13, Feyissa et al. (2019) Handb Clin Neurol. 160:103-124, Beres et al. (2017) Appl Psychophysiol Biofeedback 42(4):247-255; herein incorporated by reference.

In some embodiments, MEG is used to record magnetic fields produced by electrical currents generated in the brain. MEG detects weak magnetic fields produced by synchronized neuronal currents (i.e., ionic currents flowing in the dendrites of neurons during synaptic transmission). These weak magnetic fields can be detected using a magnetometer such as a superconducting quantum unit interference device (SQUID) or a spin exchange relaxation-free (SERF) magnetometer. For a description of MEG and methods of using MEG for recording magnetic fields associated with neuronal activity in the brain, see, e.g., Hamalainen et al. (1993) Reviews of Modern Physics. 65 (2):413-497, Baillet et al. (2017) Nat Neurosci. 20(3):327-339, Gross et al. (2019) Neuron 104(2): 189-204, Stapleton-Kotloski et al. (2018) Brain Sci. 8(8):157; herein incorporated by reference.

Patch clamping can be used to measure changes in voltage or current across cell membranes. Patch clamping can be performed, for example, using the voltage clamp technique, the current clamp technique, or the excised patch technique. In some embodiments, patch clamping comprises acquiring whole-cell recordings. Currents or voltages may be recorded through multiple channels simultaneously over one or more regions of a cell membrane. In some embodiments, patch clamping is used to monitor changes in voltage or current across cell membranes of an excitable cell. Exemplary excitable cells include, without limitation, neurons, myocytes (e.g., cardiac, skeletal, and smooth muscle cells), vascular endothelial cells, pericytes, juxtaglomerular cells, interstitial cells of Cajal, many types of epithelial cells (e.g. beta cells, alpha cells, delta cells, enteroendocrine cells, pulmonary neuroendocrine cells, and pinealocytes), glial cells (e.g., astrocytes), mechanoreceptor cells (e.g. hair cells and Merkel cells), chemoreceptor cells (e.g. glomus cells, taste receptors), some plant cells and immune cells. Excitable cells may include cells having voltage-gated ion channels, ion transporters (e.g., Na+/K+-ATPase, magnesium transporters, acid-base transporters), membrane receptors, and/or hyperpolarization-activated cyclic-nucleotide-gated channels. In some cases, patch clamping is used to monitor changes in voltage or current across the cell membrane of a neuron associated with action potentials and nerve activity.

Exemplary functional neuroimaging techniques that can be used in the practice of the subject methods include, without limitation, functional magnetic resonance imaging (fMRI), positron emission tomography (PET), functional near-infrared spectroscopy (fNIRS), single-photon emission computed tomography (SPECT), and functional ultrasound imaging (fUS). These functional neuroimaging techniques measure localized changes in cerebral blood flow and changes in the composition of blood related to neural activity. Functional neuroimaging is useful for noninvasively detecting patterns of brain activity associated with specific stimuli or tasks. In some cases, neuroimaging techniques are combined with imaging of barcoded neurons in the brain of a live subject followed by removal of a tissue specimen from the brain region where neurons were barcoded and then performing in situ sequencing of target nucleic acids in cells of the tissue specimen.

In some embodiments, fMRI is used to monitor temporal changes in blood flow associated with changes in levels of brain activity. Blood flow increases upon neuronal activation when a region of the brain is in use. Changes in brain activity can be imaged using fMRI by detection of blood oxygen-level dependent (BOLD) signals. For a description of fMRI and methods of using fMRI for imaging of brain activity, see, e.g., Ogawa, et al. (1990) Magnetic Resonance in Medicine 14 (1):68-78, Kim et al. (2002) Curr Opin Neurobiol. 12(5):607-15, Kim et al. (2012) J Cereb Blood Flow Metab. 32(7): 1188-206, Bandettini (2012) Neuroimage 62(2):575-88; Zarghami et al. (2020) Neuroimage 207:116453; Logothetis, N. K. (Jun. 12, 2008), Logothetis et al. (2008) Nature 453 (7197):869-78, and Logothetis et al. (2001) Nature. 412 (6843): 150-157; herein incorporated by reference.

In some embodiments, PET is used to monitor changes in blood flow associated with changes in neural activity. PET uses a radioactive tracer that emits positrons for imaging. Brain activity can be imaged using PET by detection of changes in blood flow, which can be measured indirectly, for example, using an oxygen-15 tracer. Areas having higher levels of radioactivity are associated with increased brain activity. For a description of PET and methods of using PET for imaging of brain activity, see, e.g., Hiura et al. (2014) J Cereb Blood Flow Metab. 34(3):389-96, Baron et al. (2012) Neuroimage 61(2):492-504, Law (2007) Dan Med Bull. 2007 November; 54(4):289-305, Ramsey et al. (1996) J Cereb Blood Flow Metab. 16(5): 755-64; herein incorporated by reference.

In some embodiments, SPECT imaging is used in brain imaging. Like PET, SPECT also uses a radioactive tracer for detecting changes in blood flow, but instead uses a tracer that emits gamma rays detectable by a gamma camera. Brain activity can be imaged by detecting changes in blood flow, for example, using Technetium 99mTc-exametazime or 99mTc-D,L-hexamethylene-propyleneamine oxime. For a description of SPECT and methods of using SPECT for imaging of brain activity, see, e.g., Cuocolo et al. (2018) Int Rev Neurobiol 141:77-96, Andersen (1989) Cerebrovasc Brain Metab Rev. 1(4):288-318, Matsuda (2001) Ann Nucl Med 15(2):85-92, Gonul et al. (2009) Int Rev Psychiatry 21(4):323-35; herein incorporated by reference.

In some embodiments, fNIRS is used to monitor changes in the composition of blood near a neural event. In particular, fNIRS can be used to detect changes in levels of oxyhemoglobin and deoxyhemoglobin using near-infrared light. Based on differences in the absorption spectra of oxyhemoglobin and deoxyhemoglobin, relative changes in hemoglobin concentration can be measured with fNIRS. Cerebral hemodynamic responses correlate with cerebral activation or deactivation. For a description of fNIRS and methods of using fNIRS for imaging of brain activity, see, e.g., Tachtsidis et al. (2020) Ann N Y Acad Sci. 1464(1):5-29, Ferrari et al. (2012) Neuroimage 63(2):921-35, Scholkmann et al. (2014) Neuroimage 85 Pt 1:6-27, Kim et al. (2017) Mol Cells 40(8):523-532; herein incorporated by reference.

In some embodiments, fUS is used to monitor localized changes in cerebral blood volume that correlate with changes in neural activity. For a description of fUS and methods of using fUS for imaging of brain activity, see, e.g., Bleton et al. (2016) BMC Med Imaging 12; 16:22, Raaij et al. (2012) Neuroimage 63(3): 1030-7; herein incorporated by reference.

In some embodiments, functional neuroimaging and/or electrophysiology techniques are used to detect brain responses when a subject is exposed to stimuli or performing tasks. Additionally, functional neuroimaging and/or electrophysiology measurements of brain activity can be taken while the subject is in a resting state (e.g., absence of stimulus or taskless) to allow brain activity to be compared to a subject's “baseline” brain state, i.e., to identify brain regions exhibiting changes in neural activity associated with specific stimuli or tasks.

In some embodiments, the methods described herein are used to evaluate changes in brain function in response to optogenetic perturbation of neural activity. In certain embodiments, optogenetics is used to induce cell-specific perturbations in the brain. For example, optogenetics can be used to excite or inhibit one or more selected neurons of interest using light, as described further below. For a description of optogenetics techniques, see, e.g., Abe et al., 2012; Desai et al., 2011; Duffy et al., 2015; Gerits et al., 2012; Kahn et al., 2013; Lee et al., 2010; Liu et al., 2015; Ohayon et al., 2013; Weitz et al., 2015; Weitz and Lee, 2013; herein incorporated by reference.

The methods described herein can also be used to evaluate changes in brain function in response to brain stimulation with electrical currents or magnetic fields that are applied to a selected brain area. For example, electrical brain stimulation (EBS) can be used to stimulate a neuron or neural network in the brain through the direct or indirect excitation of its cell membrane by using an electric current. For a description of EBS techniques, see, e.g., Aum et al. (2018) Front Biosci (Landmark Ed) 23:162-182, Tellez-Zenteno et al. (2011) Neurosurg Clin N Am. 22(4):465-75, Padberg et al. (2009) Exp. Neurol. 219:2-13, Nahas et al. (2010) Biol. Psychiatry 67:101-109, Lefaucheur et al. (2010) Exp. Neurol. 223:609-614, Levy et al. (2008) J. Neurosurg. 108:707-714, Hanajima et al. (2002) Clin. Neurophysiol. 113:635-641, Picillo et al. (2015) Brain Stimul. 8:840-842., Canavero (2014) Textbook of Cortical Brain Stimulation. Berlin: De Gruyter Open; herein incorporated by reference. Alternatively, transcranial magnetic stimulation (TMS) can be used to electrically stimulate the brain by electromagnetic induction and can be used to noninvasively stimulate specific regions of the brain. For a description of TMS techniques, see, e.g., Klomjai et al. (2015) Ann Phys Rehabil Med. 58(4):208-213, Lefaucheur (2019) Handb Clin Neurol. 160:559-580, Burke et al. (2019) Handb Clin Neurol. 163:73-92; herein incorporated by reference.

The subject methods may be applied to brain tissue from any region or regions of the brain. In certain embodiments, the one or more brain regions of interest are in the cerebrum, cerebellum, or brainstem regions of the brain. Brain regions of interest may include, without limitation, the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, and motor cortex regions of the brain.

Functional neuroimaging and/or electrophysiology data may be acquired for any type of neuron including, without limitation, unipolar neurons, bipolar neurons, multipolar neurons, Golgi I neurons, Golgi II neurons, anaxonic neurons, pseudounipolar neurons, interneurons, motor neurons, sensory neurons, afferent neurons, efferent neurons, cholinergic neurons, GABAergic neurons, glutamatergic neurons, dopaminergic neurons, serotonergic neurons, histaminergic neurons, Purkinje cells, spiny projection neurons, Renshaw cells, and granule cells, or any combination thereof.

In certain embodiments, the cell is a projection neuron. In some embodiments, the viral vector is introduced into a projection of a projection neuron, wherein retrograde transport of the viral vector delivers the viral vector to the cell body of the projection neuron. Viral vectors may be introduced into a projection of a projection neuron, for example, by stereotactic injection.

In some embodiments, the subject is an invertebrate or vertebrate including, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals. In some embodiments, the subject is a nonhuman animal. In some embodiments in which the viral vector is introduced into a neuron for investigation of neural activity, the subject may be a nonhuman subject that has a brain.

### Optogenetics

In certain embodiments, the method further comprises optogenetically modifying one or more neurons or other excitable cells in the intact tissue. For example, optogenetics can be used to allow optical control of activation (i.e., depolarization) or inhibition (i.e., hyperpolarization) of neurons that have been genetically modified to express light-responsive ion channels. In some embodiments, the light-responsive ion channel is a naturally occurring or synthetic opsin that uses a retinal-based cofactor (e.g., all-trans retinal for the microbial opsins) to respond to light. For example, light-responsive cation-conducting opsins (e.g., channelrhodopsin that conducts Ca²⁺) can be used to activate or depolarize neurons. Light-responsive anion-conducting opsins (e.g., channelrhodopsin or halorhodopsin that conduct chloride ions) or light-responsive proton conductance regulators (e.g., bacteriorhodopsin or archaerhodopsin) can be used to inhibit or hyperpolarize neurons. The levels of retinoids present in a mammalian brain are usually sufficient for expressed opsins to function without supplementation of cofactors. For a description of optogenetics and its use in controlling neural activity, see, e.g., Aravanis et al. (2007) J Neural Eng 4: S143-S156, Arenkiel et al. (2007) Neuron 54: 205-218, Boyden et al. (2005) Nat Neurosci 8: 1263-1268, Chow et al. (2010) Nature 463: 98-10, Gradinaru et al. (2007) J Neurosci 27: 14231-14238, Gradinaru et al. (2008) Brain Cell Biol 36: 129-139, Gradinaru et al. (2010) Cel/141: 1-12, Li et al. (2005) Proc Natl Acad Sci 102: 17816-17821, Lin et al. 2009. Characterization of engineered channelrhodopsin variants with improved properties and kinetics. Biophys J 96: 1803-1814, Yizhar et al. (2011) Microbial opsins: A family of single-component tools for optical control of neural activity. Cold Spring Harbor Protoc, Zhang et al. (2007) Nat Methods 4: 139-141, Zhang et al. (2006) Nat Methods 3: 785-792, Zhang et al. (2007) Nature 446: 633-639, Zhang et al. (2008) Nat Neurosci 11: 631-633; and U.S. Pat. Nos. 10,914,803; 10,589,123; 10,583,309; 10,568,516; 10,568,307; 10,538,560; 10,478,499; 10,220,092; 10,196,431; 10,087,223; 10,052,383; 9,969,783; 9,878,176; 9,855,442; 9,757,587; 9,458,208; and 8,834,546; herein incorporated by reference in their entireties.

In some embodiments, a target neuron is genetically modified to express a light-responsive ion channel that, when stimulated by an appropriate light stimulus, hyperpolarizes or depolarizes the stimulated target neuron. The term “genetic modification” refers to a permanent or transient genetic change induced in a cell following introduction into the cell of a heterologous nucleic acid (i.e., nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include the use of viral infection, transfection, conjugation, protoplast fusion, electroporation, particle gun technology, calcium phosphate precipitation, direct microinjection, and the like.

In some cases, a target cell that expresses a light-responsive polypeptide can be activated or inhibited upon exposure to light of varying wavelengths. In some cases, a target cell that expresses a light-responsive polypeptide is a neuronal cell that expresses a light-responsive polypeptide, and exposure to light of varying wavelengths results in depolarization or polarization of the neuron.

In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the excitable cell when activated by light of an activating wavelength. In some embodiments, the light-responsive polypeptide hyperpolarizes the excitable cell when activated by light of an activating wavelength.

In some cases, a light-responsive polypeptide mediates a hyperpolarizing current in the target cell it is expressed in when the cell is illuminated with light. Non-limiting examples of light-responsive polypeptides capable of mediating a hyperpolarizing current can be found, e.g., in U.S. Pat. Nos. 9,359,449; 9,175,095. Non-limiting examples of hyperpolarizing light-responsive polypeptides include NpHr, eNpHr2.0, eNpHr3.0, eNpHr3.1 or GtR3. In some cases, a light-responsive polypeptide mediates a depolarizing current in the target cell it is expressed in when the cell is illuminated with light. Non-limiting examples of depolarizing light-responsive polypeptides include “C1V1”, ChR1, VChR1, ChR2. Additional information regarding other light-responsive cation channels, anion pumps, and proton pumps can be found in U.S. Patent Application Publication No: 2009/0093403; and U.S. Pat. No. 9,359,449.

In some embodiments, the light-responsive polypeptide can be activated by blue light (e.g., in range of 490 nm-450 nm). In one embodiment, the light-responsive polypeptide can be activated by light having a wavelength of about 473 nm. In some embodiments, the light-responsive polypeptide can be activated by yellow light (e.g., in range of 590 nm-560 nm). In another embodiment, the light-responsive polypeptide can be activated by light having a wavelength of about 560 nm. In another embodiment, the light-responsive polypeptide can be activated by red light (e.g., in range of 700 nm-635 nm). In another embodiment, the light-responsive polypeptide can be activated by light having a wavelength of about 630 nm. In other embodiments, the light-responsive polypeptide can be activated by violet light (e.g., in range of 450 nm-400 nm). In one embodiment, light-responsive polypeptide can be activated by light having a wavelength of about 405 nm. In other embodiments, the light-responsive polypeptide can be activated by green light (e.g., in range of 560 nm-520 nm). In other embodiments, the light-responsive polypeptide can be activated by cyan light (e.g., in range of 520 nm-490 nm). In other embodiments, the light-responsive polypeptide can be activated by orange light (e.g., in range of 635 nm-590 nm). A person of skill in the art would recognize that each light-responsive polypeptide will have its own range of activating wavelengths.

In some cases, the regions of the brain with neurons containing a light-responsive polypeptide are illuminated using one or more optical fibers. The optical fiber may be configured in any suitable manner to direct a light emitted from a suitable source of light, e.g., a laser or light-emitting diode (LED) light source, to the region of the brain. The optical fiber may be any suitable optical fiber. In some cases, the optical fiber is a multimode optical fiber. The optical fiber may include a core defining a core diameter, where light from the light source passes through the core. The optical fiber may have any suitable core diameter. In some cases, the core diameter of the optical fiber is 10 mm or more, e.g., 20 mm or more, 30 mm or more, 40 mm or more, 50 mm or more, 60 mm or more, including 80 mm or more, and is 1,000 mm or less, e.g., 500 mm or less, 200 mm or less, 100 mm or less, including 70 mm or less. In some embodiments, the core diameter of the optical fiber is in the range of 10 to 1,000 mm, e.g., 20 to 500 mm, 30 to 200 mm, including 40 to 100 mm.

The optical fiber end that is implanted into the target region of the brain may have any suitable configuration suitable for illuminating a region of the brain with a light stimulus delivered through the optical fiber. In some cases, the optical fiber includes an attachment device at or near the distal end of the optical fiber, where the distal end of the optical fiber corresponds to the end inserted into the subject. In some cases, the attachment device is configured to connect to the optical fiber and facilitate attachment of the optical fiber to the subject, such as to the skull of the subject. Any suitable attachment device may be used. In some cases, the attachment device includes a ferrule, e.g., a metal, ceramic or plastic ferrule. The ferrule may have any suitable dimensions for holding and attaching the optical fiber.

In certain embodiments, methods of the present disclosure may be performed using any suitable electronic components to control and/or coordinate the various optical components used to illuminate the regions of the brain. The optical components (e.g., light source, optical fiber, lens, objective, mirror, and the like) may be controlled by a controller, e.g., to coordinate the light source illuminating the regions of the brain with light pulses. The controller may include a driver for the light source that controls one or more parameters associated with the light pulses, such as, but not limited to the frequency, pulse width, duty cycle, wavelength, intensity, etc. of the light pulses. The controllers may be in communication with components of the light source (e.g., collimators, shutters, filter wheels, moveable mirrors, lenses, etc.).

In some embodiments, the light-responsive polypeptides are activated by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 see, about 1.25 see, about 1.5 see, or about 2 see, inclusive, including any times in between these numbers. In some embodiments, the light-responsive polypeptides are activated by light pulses that can have a light power density of any of about 0.05 mW/mm², about 0.1 mW/mm², about 0.25 mW/mm², about 0.5 mW/mm², about 0.75 mW/mm², about 1 mW/mm², about 2 mW/mm², about 3 mW/mm², about 4 mW/mm², about 5 mW/mm², about 6 mW/mm², about 7 mW/mm², about 8 mW/mm², about 9 mW/mm², about 10 mW/mm², about 20 mW/mm², about 50 mW/mm², about 100 mW/mm², about 250 mW/mm², about 500 mW/mm², about 750 mW/mm², about 1000 mW/mm², about 1100 mW/mm², about 1200 mW/mm², about 1300 mW/mm², about 1400 mW/mm², about 1500 mW/mm², about 1600 mW/mm², about 1700 mW/mm², about 1800 mW/mm², about 1900 mW/mm², about 2000 mW/mm², about 2100 mW/mm², about 2200 mW/mm², about 2300 mW/mm², about 2400 mW/mm², about 2500 mW/mm², about 2600 mW/mm², about 2700 mW/mm², about 2800 mW/mm², about 2900 mW/mm², about 3000 mW/mm², about 3100 mW/mm², about 3100 mW/mm², about 3300 mW/mm², about 3400 mW/mm², or about 3500 mW/mm², inclusive, including any values between these numbers.

The light stimulus used to activate the light-responsive polypeptide may include light pulses characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc.

The light pulses may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 H or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some embodiments, the light pulses have a frequency in the range of 0.1 to 100,000 Hz, e.g., 1 to 10,000 Hz, 1 to 1,000 Hz, including 5 to 500 Hz, or 10 to 100 Hz.

In some cases, the two sets of light pulses are characterized by having different parameter values, such as different pulse widths, e.g. short or long. The light pulses may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The average power of the light pulse, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or
2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses may vary and may depend on the activation wavelength of the light-responsive polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

In some embodiments, the light-responsive polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-responsive protein transport to the plasma membranes of mammalian cells can be fused to the N-terminus, the C-terminus, or to both the N- and C-terminal ends of the light-responsive polypeptide. In some cases, the one or more amino acid sequence motifs which enhance light-responsive polypeptide transport to the plasma membranes of mammalian cells is fused internally within a light-responsive polypeptide. Optionally, the light-responsive polypeptide and the one or more amino acid sequence motifs may be separated by a linker. In some embodiments, the light-responsive polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In some embodiments, the signal peptide sequence in the protein can be deleted or substituted with a signal peptide sequence from a different protein.

Exemplary light-responsive polypeptides and amino acid sequence motifs that find use in the present system and method are disclosed in, e.g., U.S. Pat. Nos. 10,538,560; 10,568,307; 9,284,353; 9,359,449; and 9,365,628; herein incorporated by reference.

Light-responsive polypeptides of interest include, for example, a step function opsin (SFO)6 protein or a stabilized step function opsin (SSFO) protein that can have specific amino acid substitutions at key positions in the retinal binding pocket of the protein. See, for example, WO 2010/056970, the disclosure of which is hereby incorporated by reference in its entirety. The polypeptide may be a cation channel derived from Volvox carteri (VChR1), optionally comprising one or more amino acid substitutions, e.g., C123A; C123S; D151A, etc. A light-responsive cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1, optionally having an amino acid substitution at amino acid residue E122 or E162. In other embodiments, the light-responsive cation channel protein is a C1C2 chimeric protein derived from the ChR1 and the ChR2 proteins from Chlamydomonas reinhardti, wherein the protein is responsive to light and is capable of mediating a depolarizing current in the cell when the cell is illuminated with light. In some embodiments, a depolarizing light-responsive polypeptide is a red shifted variant of a depolarizing light-responsive polypeptide derived from Chlamydomonas reinhardtii; referred to as a “ReaChR polypeptide” or “ReaChR protein” or “ReaChR.” In some embodiments, a depolarizing light-responsive polypeptide is a SdChR polypeptide derived from Scherffelia dubia, wherein the SdChR polypeptide is capable of transporting cations across a cell membrane when the cell is illuminated with light. In some embodiments, a depolarizing light-responsive polypeptide is CnChR1, derived from Chlamydomonas noctigama, wherein the CnChR1 polypeptide is capable of transporting cations across a cell membrane when the cell is illuminated with light. In some embodiments, the light-responsive cation channel protein is a CsChrimson chimeric protein derived from a CsChR protein of Chloromonas subdivisa and CnChR1 protein from Chlamydomonas noctigama, wherein the N-terminus of the protein comprises the amino acid sequence of residues 1-73 of CsChR followed by residues 79-350 of the amino acid sequence of CnChR1; is responsive to light; and is capable of mediating a depolarizing current in the cell when the cell is illuminated with light. In some embodiments, a depolarizing light-responsive polypeptide can be, e.g., ShChR1, derived from Stigeoclonium helveticum, wherein the ShChR1 polypeptide is capable of transporting cations across a cell membrane when the cell is illuminated with light.

In some embodiments, a depolarizing light-responsive polypeptide is derived from Chlamydomonas reinhardtii (CHR1, and particularly CHR2) wherein the polypeptide is capable of transporting cations across a cell membrane when the cell is illuminated with light; and is capable of mediating a depolarizing current in the cell when the cell is illuminated with light. In some embodiments CaMKIIa-driven, humanized channelrhodopsin CHR2 H134R mutant fused to EYFP is used for optogenetic activation. The light used to activate the light-responsive cation channel protein derived from Chlamydomonas reinhardtii can have a wavelength between about 460 and about 495 nm or can have a wavelength of about 480 nm. The light-responsive cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive cation channel protein to regulate the polarization state of the plasma membrane of the cell. Additionally, the light-responsive cation channel protein can comprise one or more conservative amino acid substitutions and/or one or more non-conservative amino acid substitutions. The light-responsive proton pump protein containing substitutions, deletions, and/or insertions introduced into the native amino acid sequence suitably retains the ability to transport cations across a cell membrane. The protein may comprise various amino acid substitutions, e.g., one or more of H134R; T159C; L132C; E123A; etc. The protein may further comprise a fluorescent protein, for example, but not limited to, a yellow fluorescent protein, a red fluorescent protein, a green fluorescent protein, or a cyan fluorescent protein.

Neurons can be selectively activated or inhibited optogenetically by engineering neurons to express one or more light-responsive polypeptides configured to hyperpolarize or depolarize the neurons. Suitable light-responsive polypeptides and methods used thereof are described further below.

A light-responsive polypeptide for use in the present disclosure may be any suitable light-responsive polypeptide for selectively activating neurons of a subtype by illuminating the neurons with an activating light stimulus. In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the cell when activated by light of an activating wavelength. In some embodiments, the light-responsive polypeptide hyperpolarizes the cell when activated by light of an activating wavelength. Suitable hyperpolarizing and depolarizing polypeptides are known in the art and include, e.g., a channelrhodopsin (e.g., ChR2), variants of ChR2 (e.g., C128S, D156A, C128S+D156A, E123A, E123T), iC1C2, C1C2, GtACR2, NpHR, eNpHR3.0, C1V1, VChR1, VChR2, SwiChR, Arch, ArchT, KR2, ReaChR, ChiEF, Chronos, ChRGR, CsChrimson, and the like. In some cases, the light-responsive polypeptide includes bReaCh-ES, as described in, e.g., Rajasethupathy et al., Nature. 2015 Oct. 29;526(7575):653, which is incorporated by reference. Hyperpolarizing and depolarizing opsins have been described in various publications; see, e.g., Berndt and Deisseroth (2015) Science 349:590; Berndt et al. (2014) Science 344:420; and Guru et al. (Jul. 25, 2015) Intl. J. Neuropsychopharmacol. pp. 1-8 (PMID 26209858).

The light-responsive polypeptide may be introduced into the neurons using any suitable method. In some cases, the neurons of a subtype of interest are genetically modified to express a light-responsive polypeptide. In some cases, the neurons may be genetically modified using a viral vector, e.g., an adeno-associated viral vector, containing a nucleic acid having a nucleotide sequence that encodes the light-responsive polypeptide. The viral vector may include any suitable control elements (e.g., promoters, enhancers, recombination sites, etc.) to control expression of the light-responsive polypeptide according to neuronal subtype, timing, presence of an inducer, etc.

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non-protein coding sequence, e.g., a sequence encoding a light-reactive protein; and the like) if the promoter affects its transcription and/or expression.

Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956; see also, e.g., U.S. Pat. Nos. 6,649,811; 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor −. beta. promoter (see, e.g., Liu et al. (2620) Gene Therapy 11:52-60); a motor neuron-specific gene Hb9 promoter (see, e.g., U.S. Pat. No. 7,632,679; and Lee et al. (2620) Development 131:3295-3306); and an alpha subunit of Ca²⁺-calmodulin-dependent protein kinase II (CaMKII) promoter (see, e.g., Mayford et al. (1996) Proc. Natl. Acad. Sci. USA 93:13250). Other suitable promoters include elongation factor (EF) 1 and dopamine transporter (DAT) promoters.

In some cases, neuronal subtype-specific expression of the light-responsive polypeptide may be achieved by using recombination systems, e.g., Cre-Lox recombination, Flp-FRT recombination, etc. Cell type-specific expression of genes using recombination has been described in, e.g., Fenno et al., Nat Methods, 2014 July; 11(7): 763; and Gompf et al., Front Behav Neurosci. 2015 Jul. 2; 9:152, which are incorporated by reference herein.

In some embodiments, the vector is a recombinant adeno-associated virus (AAV) vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Virol., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, SF Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16).

Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

It is understood that one or more vectors may be administered to neural cells. If more than one vector is used, it is understood that they may be administered at the same or at different times.

Vectors for Production of mRNA Encoding a Cell Barcode

Vectors are provided for producing a mRNA transcript for barcoding cells as described herein. In some embodiments, the viral vector comprises a promoter operably linked to a sequence encoding a mRNA transcript comprising a 3′-untranslated region (3′-UTR) comprising a cell barcode and a poly-adenylation site, wherein the cell barcode is adjacent to the poly-adenylation site. In some embodiments, the mRNA transcript further comprises a coding sequence encoding a protein such as a fluorescent or bioluminescent protein or other protein of interest. The ability of constructs to produce the mRNA transcript comprising the cell barcode and any encoded proteins can be empirically determined.

Expression cassettes typically include control elements operably linked to a coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others.

Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding the mRNA transcript can be administered to a subject using standard gene delivery protocols. Methods for gene delivery are known in the art. See, e.g., U.S. Pat. Nos. 5,399,346, 5,580,859, 5,589,466. Genes can be delivered either directly to a subject or, alternatively, delivered ex vivo, to cells derived from the subject and the cells reimplanted in the subject.

A number of viral based systems can be used for delivery of a mRNA transcript into mammalian cells. These include adenoviruses, retroviruses (y-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for delivery of the mRNA transcript. Selected barcode and/or coding sequences for a protein of interest can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2): 132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems can be used for delivery of the mRNA transcript. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the mRNA transcript is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the mRNA transcript include those derived from the pox family of viruses, including vaccinia virus and avian poxvirus. By way of example, vaccinia virus recombinants expressing the mRNA transcript can be constructed as follows. The DNA encoding the particular mRNA transcript is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the mRNA transcript. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the mRNA transcript carrying the barcode. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., Tₘ. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the mRNA transcript in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into the mRNA carrying the cell barcode, and coding sequences (e.g., for a fluorescent or bioluminescent protein or other protein of interest) may then translated into protein by the host translational machinery. The method provides for high level, transient, cytoplasmic production of large quantities of mRNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The mRNA transcript (or a nucleic acid encoding it) can also be delivered without a viral vector. For example, a synthetic mRNA transcript can be packaged in liposomes prior to delivery to the subject or to cells derived therefrom. Lipid encapsulation is generally accomplished using liposomes which are able to stably bind or entrap and retain nucleic acid. The ratio of condensed DNA/RNA to lipid preparation can vary but will generally be around 1:1 (mg DNA/RNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations may include cationic (positively charged), anionic (negatively charged) and neutral preparations, with cationic liposomes particularly preferred. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N, N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, AL), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in Methods of Immunology (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The RNA, DNA, and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2): 197-210, 1997; O'Hagan D. T., et al., Vaccine 11(2): 149-54, 1993.

Furthermore, other particulate systems and polymers can be used for the in vivo or ex vivo delivery of the mRNA transcript. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering the mRNA transcript or a vector encoding it. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors encoding the mRNA transcript carrying the cell barcode are formulated into compositions for delivery to a subject. The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of the mRNA transcript carrying the cell barcode in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

### In Situ Sequencing

In situ sequencing may be performed, for example, using Spatially-resolved Transcript Amplicon Readout Mapping (STARmap) technique. For a description of the original STARmap technique, see, e.g., International Patent Application Publication No. WO2019/199579 A1 and Wang et al. (2018) Science 361(6400):eaat5691; herein incorporated by reference in their entireties. Modified versions of STARmap may also be used for in situ sequencing, such as described in International Patent Application Publication No. WO/2021/076770 A1 and in the co-owned International Patent Application entitled “NEXT-GENERATION VOLUMETRIC IN SITU SEQUENCING,” filed even date herewith, the disclosures of which are hereby incorporated by reference herein in their entireties. STARmap methods and variations thereof utilize image-based in situ nucleic acid (DNA and/or RNA) sequencing technology using a sequencing-by-ligation process, specific signal amplification, hydrogel-tissue chemistry to turn biological tissue into a transparent sequencing chip, and associated data analysis pipelines to spatially-resolve highly-multiplexed gene detection at a subcellular and cellular level. In some other aspects, the methods disclosed herein include spatially sequencing (e.g. reagents, chips or services) for biomedical research and clinical diagnostics (e.g. cancer, bacterial infection, viral infection, etc.) with single-cell and/or single-molecule sensitivity.

In some embodiments, in situ gene sequencing of a target nucleic acid in a cell in an intact tissue is performed using a method comprising: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon; (f) reiterating step (e); and (g) imaging the one or more hydrogel-embedded amplicons to determine in situ a gene sequence of the target nucleic acid in the cell in the intact tissue.

In certain embodiments, the target nucleic acid is the mRNA transcript comprising the 3′-untranslated region (3′-UTR) comprising the cell barcode and the poly-adenylation site that was introduced into the cell with a viral vector, wherein imaging is used to determine the sequence of the cell barcode. In some embodiments, the length of the cell barcode sequence is sufficient to allow at least one pair of oligonucleotide primers to bind to the cell barcode sequence, wherein the first complementarity region of the first oligonucleotide is complementary to a first portion of the barcode sequence, wherein the second complementary region of the second oligonucleotide is complementary to a second portion of the barcode sequence, and wherein the first portion of the barcode sequence is adjacent to the second portion of the barcode sequence. In some embodiments, the length of the cell barcode sequence is sufficient to allow at least two pairs of oligonucleotide primers to bind to the cell barcode sequence. In some embodiments, the length of the cell barcode sequence is sufficient to allow at least four pairs of oligonucleotide primers to bind to the cell barcode sequence. In some embodiments, the length of the cellular barcode sequence in the mRNA transcript is sufficient for 1 to 5 pairs of oligonucleotide primers to bind to the cellular barcode sequence, including any number within this range such as 1, 2, 3, 4, or 5 pairs of oligonucleotide primers, wherein the oligonucleotide primers have complementarity regions that are complementary to a portion of the cellular barcode sequence. In some embodiments, the cell barcode sequence has a length of at least 30 nucleotides, at least 40 nucleotides, at least 50 nucleotides, or at least 60 nucleotides.

In certain embodiments, the method further comprises contacting the fixed and permeabilized intact tissue with a gel adaptor oligonucleotide that binds to the first oligonucleotide, wherein the gel adaptor oligonucleotide comprises a nucleotide modification at the 5′ end that links the gel adapter to the hydrogel during gelation. In some embodiments, the modification comprises an acrydite group. In some embodiments, the first oligonucleotide further comprises a common binding site for the gel adaptor oligonucleotide. In some embodiments, the common binding site for the gel adaptor oligonucleotide is adjacent to the first complementarity region of the first oligonucleotide.

In certain embodiments, the method further comprises barcoding a cell by contacting the cell with: a first probe comprising a 5′-amine modification or a 5′-biotin modification, a common gel adaptor complementary sequence that hybridizes with the gel adaptor oligonucleotide, and a unique barcode sequence; and a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe. In some embodiments, the second probe is a padlock probe. In some embodiments, a plurality of first probes and second probes are used to barcode a plurality of cells in the intact tissue, wherein each first probe has a different unique barcode sequence.

The methods disclosed herein also provide for a method of screening a candidate agent to determine whether the candidate agent modulates gene expression of a nucleic acid in a cell in an intact tissue by performing a method described herein to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

### Specific Amplification of Nucleic Acids Via Intramolecular Ligation (SNAIL)

In some embodiments, in situ sequencing is performed using Specific Amplification of Nucleic Acids via Intramolecular Ligation (SNAIL), an efficient approach for generating cDNA libraries from cellular RNAs in situ. In certain embodiments, the methods of the invention include contacting a fixed and permeabilized intact tissue with at least a pair of oligonucleotide primers under conditions to allow for specific hybridization, wherein the pair of primers includes a first oligonucleotide and a second oligonucleotide.

More generally, the nucleic acid present in a cell of interest in a tissue serves as a scaffold for an assembly of a complex that includes a pair of primers, referred to herein as a first oligonucleotide and a second oligonucleotide. In some embodiments, the contacting the fixed and permeabilized intact tissue includes hybridizing the pair of primers to the same target nucleic acid. In some embodiments, the target nucleic acid is RNA. In such embodiments, the target nucleic acid is mRNA. In other embodiments, the target nucleic acid is DNA.

As used herein, the terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

### SNAIL Oligonucleotide Primers

In the subject methods, the SNAIL oligonucleotide primers include at least a first oligonucleotide and a second oligonucleotide; wherein each of the first oligonucleotide and the second oligonucleotide includes a first complementarity region, a second complementarity region, and a third complementarity region; wherein the second oligonucleotide further includes a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, and wherein the first complementarity region of the first oligonucleotide is adjacent to the second complementarity region of the second oligonucleotide. In an alternative embodiment, the second oligonucleotide is a closed circular molecule, and a ligation step is omitted.

The present disclosure provides methods where the contacting a fixed and permeabilized tissue includes hybridizing a plurality of oligonucleotide primers having specificity for different target nucleic acids. In some embodiments, the methods include a plurality of first oligonucleotides, including, but not limited to, 5 or more first oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of first oligonucleotides, including, but not limited to, 15 or more first oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of second oligonucleotides, including, but not limited to, 5 or more second oligonucleotides, e.g., 8 or more, 10 or more, 12 or more, 15 or more, 18 or more, 20 or more, 25 or more, 30 or more, 35 or more. In some embodiments, a method of the present disclosure includes a plurality of second oligonucleotides including, but not limited to, 15 or more second oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids. The primers are typically denatured prior to use, typically by heating to a temperature of at least about 50° C., at least about 60° C., at least about 70° C., at least about 80° C., and up to about 99° C., up to about 95° C., up to about 90° C.

In certain embodiments, the target nucleic acid is the mRNA transcript comprising the cellular barcode that was introduced into the cell using a viral vector. In some embodiments, the length of the cellular barcode sequence in the mRNA transcript is sufficient for at least 1, at least 2, at least 3, or at least 4 pairs of SNAIL oligonucleotide primers to bind to the cellular barcode sequence. In some embodiments, the length of the cellular barcode sequence in the mRNA transcript is sufficient for 1 to 5 SNAIL oligonucleotide primers to bind to the cellular barcode sequence, including any number within this range such as 1, 2, 3, 4, or 5 pairs of SNAIL oligonucleotide primers, wherein the SNAIL oligonucleotide primers have complementarity regions that are complementary to a portion of the cellular barcode sequence.

In some embodiments, the primers are denatured by heating before contacting the sample. In certain aspects, the melting temperature (Tₘ) of oligonucleotides is selected to minimize ligation in solution. The “melting temperature” or “Tₘ” of a nucleic acid is defined as the temperature at which half of the helical structure of the nucleic acid is lost due to heating or other dissociation of the hydrogen bonding between base pairs, for example, by acid or alkali treatment, or the like. The Tₘ of a nucleic acid molecule depends on its length and on its base composition. Nucleic acid molecules rich in GC base pairs have a higher Tₘ than those having an abundance of AT base pairs. Separated complementary strands of nucleic acid spontaneously reassociate or anneal to form duplex nucleic acid when the temperature is lowered below the Tₘ. The highest rate of nucleic acid hybridization occurs approximately 25 degrees C. below the Tₘ. The Tₘ may be estimated using the following relationship: Tₘ=69.3+0.41(GC) % (Marmur et al. (1962) J. Mol. Biol. 5:109-118).

In certain embodiments, the plurality of second oligonucleotides includes a padlock probe. In some embodiments, the probe includes a detectable label that can be measured and quantitated. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

In some embodiments, the one or more first oligonucleotides and second oligonucleotides bind to a different region of the target nucleic acid, or target site. In a pair, each target site is different, and the target sites are adjacent sites on the target nucleic acid, e.g. usually not more than 15 nucleotides distant, e.g. not more than 10, 8, 6, 4, or 2 nucleotides distant from the other site, and may be contiguous sites. Target sites are typically present on the same strand of the target nucleic acid in the same orientation. Target sites are also selected to provide a unique binding site, relative to other nucleic acids present in the cell. Each target site is generally from about 19 to about 25 nucleotides in length, e.g. from about 19 to 23 nucleotides, from about 19 to 21 nucleotides, or from about 19 to 20 nucleotides. The pair of first and second oligonucleotides are selected such that each oligonucleotide in the pair has a similar melting temperature for binding to its cognate target site, e.g. the Tₘ may be from about 50° C., from about 52° C., from about 55° C., from about 58°, from about 62° C., from about 65° C., from about 70° C., or from about 72° C. The GC content of the target site is generally selected to be no more than about 20%, no more than about 30%, no more than about 40%, no more than about 50%, no more than about 60%, no more than about 70%,

In some embodiments, the first oligonucleotide includes a first, second, and third complementarity region. The target site of the first oligonucleotide may refer to the first complementarity region. As summarized above, the first complementarity region of the first oligonucleotide may have a length of 19-25 nucleotides. In certain aspects, the second complementarity region of the first oligonucleotide has a length of 3-10 nucleotides, including, e.g., 4-8 nucleotides or 4-7 nucleotides. In some aspects, the second complementarity region of the first oligonucleotide has a length of 6 nucleotides. In some embodiments, the third complementarity region of the first oligonucleotide likewise has a length of 6 nucleotides. In such embodiments, the third complementarity region of the first oligonucleotide has a length of 3-10 nucleotides, including, e.g., 4-8 nucleotides or 4-7 nucleotides.

In some embodiments, second first oligonucleotide includes a first, second, and third complementarity region. The target site of the second oligonucleotide may refer to the second complementarity region. As summarized above, the second complementarity region of the second oligonucleotide may have a length of 19-25 nucleotides. In certain aspects, the first complementarity region of the first oligonucleotide has a length of 3-10 nucleotides, including, e.g., 4-8 nucleotides or 4-7 nucleotides. In some aspects, the first complementarity region of the first oligonucleotide has a length of 6 nucleotides. In some aspects, the first complementarity region of the second oligonucleotide includes the 5′ end of the second oligonucleotide. In some embodiments, the third complementarity region of the second oligonucleotide likewise has a length of 6 nucleotides. In such embodiments, the third complementarity region of the second oligonucleotide has a length of 3-10 nucleotides, including, e.g., 4-8 nucleotides or 4-7 nucleotides. In further embodiments, the third complementarity region of the second oligonucleotide includes the 3′ end of the second oligonucleotide. In some embodiments, the first complementarity region of the second oligonucleotide is adjacent to the third complementarity region of the second oligonucleotide.

In some aspects, the second oligonucleotide includes a barcode sequence, wherein the barcode sequence of the second oligonucleotide provides barcoding information for identification of the target nucleic acid. The term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out amplification with an oligonucleotide that contains a region including the barcode sequence and a region that is complementary to the target nucleic acid such that the barcode sequence is incorporated into the final amplified target nucleic acid product (i.e., amplicon).

In some embodiments, the first oligonucleotide further comprises a common binding site for a gel adaptor oligonucleotide. The gel adaptor oligonucleotide comprises a functional attachment modification at its 5′ end, such as acrydite, such that the first oligonucleotide is covalently linked via the gel adaptor oligonucleotide to the hydrogel during gelation. The use of a gel adaptor helps to retain amplicons grown from the 3′ end of the first oligonucleotide in a gel, without the need for the first oligonucleotide to have a 5′ modification itself. In some embodiments, the common binding site for the gel adaptor oligonucleotide is adjacent to the first complementarity region of the first oligonucleotide.

### Tissue

As described herein, the methods disclosed include in situ sequencing technology of an intact tissue by at least contacting a fixed and permeabilized intact tissue with at least a pair of oligonucleotide primers under conditions to allow for specific hybridization. Tissue specimens suitable for use with the methods described herein generally include any type of tissue specimens collected from living or dead subjects, such as, e.g., biopsy specimens and autopsy specimens, of which include, but are not limited to, epithelium, muscle, connective, and nervous tissue. Tissue specimens may be collected and processed using the methods described herein and subjected to microscopic analysis immediately following processing, or may be preserved and subjected to microscopic analysis at a future time, e.g., after storage for an extended period of time. In some embodiments, the methods described herein may be used to preserve tissue specimens in a stable, accessible and fully intact form for future analysis. In some embodiments, the methods described herein may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In other embodiments, the intact tissue is a thick slice with a thickness of 50-200 μm, including, but not limited to, e.g., 50-150 μm, 50-100 μm, or 50-80 μm.

Aspects of the invention include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A sample can be contacted by a fixation reagent for a period of time in a range of from 5 minutes to 24 hours, e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes.

A sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° ° C., and −18 to −22° C. In some instances a sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

A sample can be contacted by a permeabilization reagent for a wide range of times, which can depend on the temperature, the nature of the sample, and on the permeabilization reagent(s). For example, a sample can be contacted by a permeabilization reagent for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. A sample can be contacted by a permeabilization reagent at various temperatures, depending on the protocol and the reagent used. For example, in some instances a sample can be contacted by a permeabilization reagent at a temperature ranging from −82° C. to 55° C., where specific ranges of interest include, but are not limited to: 50 to 54° C., 40 to 44° C., 35 to 39ºC, 28 to 32° C., 20 to 26° ° C., 0 to 6° C., −18 to −22° C., and −78 to −82° C. In some instances a sample can be contacted by a permeabilization reagent at a temperature of −80° C., −20° C., 4° C., room temperature (22-25° C.), 30° C., 37° C., 42° ° C., or 52° C.

In some embodiments, a sample is contacted with an enzymatic permeabilization reagent. Enzymatic permeabilization reagents that permeabilize a sample by partially degrading extracellular matrix or surface proteins that hinder the permeation of the sample by assay reagents. Contact with an enzymatic permeabilization reagent can take place at any point after fixation and prior to target detection. In some instances the enzymatic permeabilization reagent is proteinase K, a commercially available enzyme. In such cases, the sample is contacted with proteinase K prior to contact with a post-fixation reagent. Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For example, a sample can be contacted by proteinase K for 30 or less minutes, 25 or less minutes, 20 or less minutes, 15 or less minutes, 10 or less minutes, 5 or less minutes, or 2 or less minutes. A sample can be contacted by 1 ug/ml or less, 2 ug/m or less, 4 ug/ml or less, 8 ug/ml or less, 10 ug/ml or less, 20 ug/ml or less, 30 ug/ml or less, 50 ug/ml or less, or 100 ug/ml or less proteinase K. A sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a sample can be contacted by proteinase K at a temperature of 4° C., room temperature (22-25° C.), 30° C., 37° ° C., 42° C., or 52° C. In some embodiments, a sample is not contacted with an enzymatic permeabilization reagent. In some embodiments, a sample is not contacted with proteinase K. Contact of an intact tissue with at least a fixation reagent and a permeabilization reagent results in the production of a fixed and permeabilized tissue.

### Ligase

In some embodiments, the methods disclosed include adding ligase to ligate the second oligonucleotide and generate a closed nucleic acid circle. In some embodiments, the adding ligase includes adding DNA ligase. In alternative embodiments, the second oligonucleotide is provided as a closed nucleic acid circle, and the step of adding ligase is omitted. In certain embodiments, ligase is an enzyme that facilitates the sequencing of a target nucleic acid molecule.

The term “ligase” as used herein refers to an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1 0.2 (NAD+-dependent ligases), EC 6.5.1 0.3 (RNA ligases). Specific examples of ligases include bacterial ligases such as E. coli DNA ligase and Taq DNA ligase, Ampligase® thermostable DNA ligase (Epicentre® Technologies Corp., part of Illumina®, Madison, Wis.) and phage ligases such as T3 DNA ligase, T4 DNA ligase and T7 DNA ligase and mutants thereof. The method relies on the specificity of the ligase, wherein a ligase can be used that does not tolerate mismatched sequences.

### Rolling Circle Amplification

In some embodiments, the methods of the invention include the step of performing rolling circle amplification in the presence of a nucleic acid molecule, wherein the performing includes using the second oligonucleotide as a template and the first oligonucleotide as a primer for a polymerase to form one or more amplicons. In such embodiments, a single-stranded, circular polynucleotide template is formed by ligation of the second nucleotide, which circular polynucleotide includes a region that is complementary to the first oligonucleotide. Upon addition of a DNA polymerase in the presence of appropriate dNTP precursors and other cofactors, the first oligonucleotide is elongated by replication of multiple copies of the template. This amplification product can be readily detected by binding to a detection probe. In some embodiments, the polymerase is preincubated without dNTPs to allow the polymerase to penetrate the sample uniformly before performing rolling circle amplification.

In some embodiments, only when a first oligonucleotide and second oligonucleotide hybridize to the same target nucleic acid molecule, the second oligonucleotide can be circularized and rolling-circle amplified to generate a cDNA nanoball (i.e., amplicon) containing multiple copies of the cDNA. The term “amplicon” refers to the amplified nucleic acid product of a PCR reaction or other nucleic acid amplification process. In some embodiments, amine-modified nucleotides are spiked into the rolling circle amplification reaction.

Techniques for rolling circle amplification are known in the art (see, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1: 1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

In certain aspects, the nucleic acid molecule includes an amine-modified nucleotide. In such embodiments, the amine-modified nucleotide includes an acrylic acid N-hydroxysuccinimide moiety modification. Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

### Amplicon Embedding in a Tissue-hydrogel Setting

In some embodiments, the methods disclosed include embedding one or more amplicons in the presence of hydrogel subunits to form one or more hydrogel-embedded amplicons. The hydrogel-tissue chemistry described includes covalently attaching nucleic acids to in situ synthesized hydrogel for tissue clearing, enzyme diffusion, and multiple-cycle sequencing while an existing hydrogel-tissue chemistry method cannot. In some embodiments, to enable amplicon embedding in the tissue-hydrogel setting, amine-modified nucleotides are spiked into the rolling circle amplification reaction, functionalized with an acrylamide moiety using acrylic acid N-hydroxysuccinimide esters, and copolymerized with acrylamide monomers to form a hydrogel.

As used herein, the terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” mean hydrophilic monomers, prepolymers, or polymers that can be crosslinked, or “polymerized”, to form a three-dimensional (3D) hydrogel network. Without being bound by any scientific theory, it is believed that this fixation of the biological specimen in the presence of hydrogel subunits crosslinks the components of the specimen to the hydrogel subunits, thereby securing molecular components in place, preserving the tissue architecture and cell morphology.

In some embodiments, the embedding includes copolymerizing the one or more amplicons with acrylamide. As used herein, the term “copolymer” describes a polymer which contains more than one type of subunit. The term encompasses polymer which include two, three, four, five, or six types of subunits.

In certain aspects, the embedding includes clearing the one or more hydrogel-embedded amplicons wherein the target nucleic acid is substantially retained in the one or more hydrogel-embedded amplicons. In such embodiments, the clearing includes substantially removing a plurality of cellular components from the one or more hydrogel-embedded amplicons. In some other embodiments, the clearing includes substantially removing lipids and/or proteins from the one or more hydrogel-embedded amplicons. As used herein, the term “substantially” means that the original amount present in the sample before clearing has been reduced by approximately 70% or more, such as by 75% or more, such as by 80% or more, such as by 85% or more, such as by 90% or more, such as by 95% or more, such as by 99% or more, such as by 100%.

In some embodiments, clearing the hydrogel-embedded amplicons includes performing electrophoresis on the specimen. In some embodiments, the amplicons are electrophoresed using a buffer solution that includes an ionic surfactant. In some embodiments, the ionic surfactant is sodium dodecyl sulfate (SDS). In some embodiments, the specimen is electrophoresed using a voltage ranging from about 10 to about 60 volts. In some embodiments, the specimen is electrophoresed for a period of time ranging from about 15 minutes up to about 10 days. In some embodiments, the methods further involve incubating the cleared specimen in a mounting medium that has a refractive index that matches that of the cleared tissue. In some embodiments, the mounting medium increases the optical clarity of the specimen. In some embodiments, the mounting medium includes glycerol.

### Sequencing

In some embodiments, SEDAL, SEDAL2, or SCAL sequencing-by-ligation methods are used. The methods disclosed herein include the step of contacting one or more hydrogel-embedded amplicons having a barcode sequence with a pair of primers under conditions to allow for ligation, wherein the pair of primers include a third oligonucleotide and a fourth oligonucleotide, wherein ligation only occurs when both the third oligonucleotide and the fourth oligonucleotide ligate to the same amplicon. In some embodiments, the third oligonucleotide is configured to decode bases and the fourth oligonucleotide is configured to convert decoded bases into a signal. In some aspects, the signal is a fluorescent signal. In exemplary aspects, the contacting the one or more hydrogel-embedded amplicons having the barcode sequence with a pair of primers under conditions to allow for ligation involves each of the third oligonucleotide and the fourth oligonucleotide ligating to form a stable product for imaging only when a perfect match occurs. In certain aspects, the mismatch sensitivity of a ligase enzyme is used to determine the underlying sequence of the target nucleic acid molecule.

Inclusion of a polyethylene glycol (PEG) polymer in the sequencing ligation mixture substantially accelerates signal addition onto target nucleic acids. Exemplary PEG polymers have molecular weights ranging from 300 g/mol to 10,000,000 g/mol. In some embodiments, a PEG 6000 polymer is present during ligation of the third and fourth oligonucleotides.

In some embodiments, the contacting the one or more hydrogel-embedded amplicons occurs two times or more, including, but not limited to, e.g., three times or more, four times or more, five times or more, six times or more, or seven times or more. In certain embodiments, the contacting the one or more hydrogel-embedded amplicons occurs four times or more for thin tissue specimens. In other embodiments, the contacting the one or more hydrogel-embedded amplicons occurs six times or more for thick tissue specimens. In some embodiments, one or more amplicons can be contacted by a pair of primers for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. In some embodiments, the methods are performed at room temperature for preservation of tissue morphology with low background noise and error reduction. In some embodiments, the contacting the one or more hydrogel-embedded amplicons includes eliminating error accumulation as sequencing proceeds.

Specimens prepared using the subject methods may be analyzed by any of a number of different types of microscopy, for example, optical microscopy (e.g. bright field, oblique illumination, dark field, phase contrast, differential interference contrast, interference reflection, epifluorescence, confocal, etc., microscopy), laser microscopy, electron microscopy, and scanning probe microscopy. In some aspects, a non-transitory computer readable medium transforms raw images acquired through microscopy of multiple rounds of in situ sequencing first into decoded gene identities and spatial locations and then analyzes the per-cell composition of gene expression.

The term “perfectly matched”, when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, peptide nucleic acids (PNAs), and the like, that may be employed. A “mismatch” in a duplex between two oligonucleotides means that a pair of nucleotides in the duplex fails to undergo Watson-Crick bonding.

In some embodiments, the method includes a plurality of third oligonucleotides, including, but not limited to, 5 or more third oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of third oligonucleotides, including, but not limited to, 15 or more third oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of fourth oligonucleotides, including, but not limited to, 5 or more fourth oligonucleotides, e.g., 8 or more, 10 or more, 12 or more, 15 or more, 18 or more, 20 or more, 25 or more, 30 or more, 35 or more. In some embodiments, a method of the present disclosure includes a plurality of fourth oligonucleotides including, but not limited to, 15 or more fourth oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids.

In certain embodiments, sequencing is performed with a ligase with activity hindered by base mismatches, a third oligonucleotide, and a fourth oligonucleotide. The term “hindered” in this context refers to activity of a ligase that is reduced by approximately 20% or more, such as by 25% or more, such as by 50% or more, such as by 75% or more, such as by 90% or more, such as by 95% or more, such as by 99% or more, such as by 100%. In some embodiments, the third oligonucleotide has a length of 5-15 nucleotides, including, but not limited to, 5-13 nucleotides, 5-10 nucleotides, or 5-8 nucleotides. In some embodiments, the Tₘ of the third oligonucleotide is at room temperature (22-25° C.). In some embodiments, the third oligonucleotide is degenerate, or partially thereof. In some embodiments, the fourth oligonucleotide has a length of 5-15 nucleotides, including, but not limited to, 5-13 nucleotides, 5-10 nucleotides, or 5-8 nucleotides. In some embodiments, the Tₘ of the fourth oligonucleotide is at room temperature (22°−25° C.). After each cycle of sequencing corresponding to a base readout, the fourth oligonucleotides may be stripped, which eliminates error accumulation as sequencing proceeds. In some embodiments, the fourth oligonucleotides are stripped by formamide.

In some embodiments, sequencing involves the washing of the third oligonucleotide and the fourth oligonucleotide to remove unbound oligonucleotides, thereafter revealing a fluorescent product for imaging. In certain exemplary embodiments, a detectable label can be used to detect one or more nucleotides and/or oligonucleotides described herein. In certain embodiments, a detectable label can be used to detect the one or more amplicons. Examples of detectable markers include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorimidases, phosphatases, peroxidases, cholinesterases and the like. Identifiable markers also include radioactive compounds such as 1251, 35S, 14C, or 3H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5, 151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444, 143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Metallic silver or gold particles may be used to enhance signal from fluorescently labeled nucleotide and/or oligonucleotide sequences (Lakowicz et al. (2003) Bio Techniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5, 198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, an antioxidant compound is included in the washing and imaging buffers (i.e., “anti-fade buffers”) to reduce photobleaching during fluorescence imaging. Exemplary antioxidants include, without limitation, Trolox (6-hydroxy-2,5,7,8-tetramethylchroman-2-carboxylic acid) and Trolox-quinone, propyl-gallate, tertiary butylhydroquinone, butylated hydroxyanisole, butylated hydroxytoluene, glutathione, ascorbic acid, and tocopherols. Such antioxidants have an antifade effect on fluorophores. That is, the antioxidant reduces photobleaching during tiling, greatly enhances the signal-to-noise ratio (SNR) of sensitive fluorophores, and enables higher SNR imaging of thicker samples. For a fixed exposure time, including an antioxidant increases the SNR by increasing the concentration of the non-bleached fluorophore during exposure to light. Including an antioxidant also removes the diminishing returns of longer exposure times (caused by the limited fluorophore lifetime before photobleaching), providing for increased SNR by allowing increased exposure times.

In addition, fluorophore cleavage from probes or probe stripping can be used to eliminate signal carryover from one round to the next when multiple sequencing cycles are used. For example, fluorophores can be stripped off with formamide. Alternatively, thiol-linked dyes can be used having a disulfide linkage between the fluorophore and an oligonucleotide probe, which enables cleavage of the fluorophore from the oligonucleotide probe in a reducing environment. Exemplary disulfide reducing agents, which can be used for cleaving disulfide bonds include, without limitation, tris(2-carboxyethyl)phosphine (TCEP), dithiothreitol (DTT), and ß-mercaptoethanol (BME). Following fluorescence imaging during a sequencing round, a stripping agent and/or a reducing agent is added, and subsequent washing steps remove the diffusive fluorescent signal before performing another round of sequencing.

A sequencing cycle for SCAL or SEDAL2 optionally begins with a brief sample wash, before proceeding to the first signal addition. For SCAL sequencing, depending on whether sequential or combinatorial encoding is being used for a particular round, the corresponding set of third and fourth oligonucleotides and their round-specific competitors are added and ligated. In combinatorial encodings, the third oligonucleotide for a given position x is added, plus a set of fluorescently labeled dibase-encoding oligonucleotides, plus a competitor oligonucleotide for the previous position that was labeled (unless it is the first round of labeling, in which case competitor oligonucleotide is omitted). In sequential encodings, the third oligonucleotide for a given round x, a 4-channel fluorophore mixture, and a round x-1 competitor oligonucleotide are added, except if it is the first round of labeling. The presence of PEG in the sequencing ligation mixture substantially accelerates the signal addition onto the target. Following incubation of the sample in imaging buffer, the sample is imaged, and briefly rinsed before proceeding to the next sequencing cycle.

For SEDAL2, the same oligonucleotide/ligation mixture is used as described above during the signal addition phase, except competitor oligonucleotides are omitted. Following sample addition, washing, imaging buffer addition, and imaging as described above, SEDAL2 includes a separate phase for signal removal, in which signals are either stripped off with a formamide-containing stripping solution or if thiol-linked dyes are used for sequential encoding fluorescently labeled oligonucleotides, a cleaving solution containing a disulfide reducing agent (e.g., TCEP). Samples are subsequently washed before proceeding to the next round of signal addition.

In certain embodiments, sequencing the barcode of the mRNA transcript comprises performing single-cell 3′-RNA sequencing of the mRNA transcript. RNA with 3′ polyA tails can be isolated from a cell by poly(A) selection using poly(T) oligomers. In some embodiments, the poly(T) oligomers are bound to a solid support. For example, the use of magnetic beads with immobilized poly(T) oligomers attached to the surface of the bead allows magnetic separation techniques to be used to isolate RNA with 3′ poly(A) tails from heterogeneous mixtures. In some embodiments, the RNA is reverse transcribed to generate cDNA for sequencing using a reverse transcriptase. In other embodiments, the RNA is directly sequenced using single-molecule real-time RNA sequencing. Either the nanopore sequencing platform of Oxford Nanopore Technologies (Oxford, United Kingdom) or the IsoSeq sequencing platform of Pacific Biosciences (Menlo Park, CA) can be used, for example, to directly sequence a mRNA transcript carrying a cell barcode. In some cases, next-generation sequencing (NGS) is performed with short reads, for example, with sequencing reads starting from the 3′ poly(A) tail and reading through the barcode.

Cell barcodes for endogenous RNA targets can be combined with exogenous barcodes carried by mRNA introduced by a viral vector for multi-feature integration. For example, a cell barcode associated with measured morphological or functional characteristics can be tied to a particular cell identified by its corresponding barcode. Morphological or functional characteristics may be measured using various methods such as, but not limited to, performing gene expression profiling, microscopy (e.g., confocal microscopy, atomic force microscopy, super-resolution microscopy, light-sheet microscopy, two-photon microscopy, or fluorescence microscopy), calcium imaging, electrophysiology measurements (e.g., patch clamping, electroencephalography (EEG), and magnetoencephalography (MEG)), functional neuroimaging (e.g., functional magnetic resonance imaging (fMRI), positron emission tomography (PET), functional near-infrared spectroscopy (fNIRS), single-photon emission computed tomography (SPECT), or functional ultrasound imaging (fUS). functional magnetic resonance imaging (fMRI)), migration assays, axonal growth and pathfinding assays, phagocytosis assays, enzymatic assays, cell receptor assays, ion channel assays, signal transduction assays, or cell secretion assays, or any combination thereof. In some embodiments, in situ gene sequencing data is combined with one or more, two or more, three or more, four or more, or five or more other types of experimental measurements, wherein cell barcoding is used to match the experimental data obtained by these measurements with the in situ sequencing data for an individual cell in the tissue.

### Cells

Methods disclosed herein include a method for in situ gene sequencing of a target nucleic acid in a cell in an intact tissue. In certain embodiments, the cell is present in a population of cells. In certain other embodiments, the population of cells includes a plurality of cell types including, but not limited to, excitatory neurons, inhibitory neurons, and non-neuronal cells. Cells for use in the assays of the invention can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment of the invention, the cells are mammalian cells, e.g. complex cell populations such as naturally occurring tissues, for example blood, liver, pancreas, neural tissue, bone marrow, skin, and the like. Some tissues may be disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, ThO T cells, cytotoxic T cells; B cells, pre-B cells, etc.; monocytes; dendritic cells; neutrophils; and macrophages; natural killer cells; mast cells, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, VA) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they may be dissociated from the substrate that they are adhered to, and from other cells, in a manner that maintains their ability to recognize and bind to probe molecules.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

### Imaging

The methods disclosed include imaging the one or more hydrogel-embedded amplicons using any of a number of different types of microscopy, e.g., confocal microscopy, two-photon microscopy, light-field microscopy, intact tissue expansion microscopy, and/or CLARITY™-optimized light sheet microscopy (COLM).

Bright field microscopy is the simplest of all the optical microscopy techniques. Sample illumination is via transmitted white light, i.e. illuminated from below and observed from above. Limitations include low contrast of most biological samples and low apparent resolution due to the blur of out of focus material. The simplicity of the technique and the minimal sample preparation required are significant advantages.

In oblique illumination microscopy, the specimen is illuminated from the side. This gives the image a 3-dimensional appearance and can highlight otherwise invisible features. A more recent technique based on this method is Hoffmann's modulation contrast, a system found on inverted microscopes for use in cell culture. Though oblique illumination suffers from the same limitations as bright field microscopy (low contrast of many biological samples; low apparent resolution due to out of focus objects), it may highlight otherwise invisible structures.

Dark field microscopy is a technique for improving the contrast of unstained, transparent specimens. Dark field illumination uses a carefully aligned light source to minimize the quantity of directly-transmitted (unscattered) light entering the image plane, collecting only the light scattered by the sample. Dark field can dramatically improve image contrast (especially of transparent objects) while requiring little equipment setup or sample preparation. However, the technique suffers from low light intensity in final image of many biological samples, and continues to be affected by low apparent resolution.

Phase contrast is an optical microscopy illumination technique that converts phase shifts in light passing through a transparent specimen to brightness changes in the image. In other words, phase contrast shows differences in refractive index as difference in contrast. The phase shifts themselves are invisible to the human eye, but become visible when they are shown as brightness changes.

In differential interference contrast (DIC) microscopy, differences in optical density will show up as differences in relief. The system consists of a special prism (Nomarski prism, Wollaston prism) in the condenser that splits light in an ordinary and an extraordinary beam. The spatial difference between the two beams is minimal (less than the maximum resolution of the objective). After passage through the specimen, the beams are reunited by a similar prism in the objective. In a homogeneous specimen, there is no difference between the two beams, and no contrast is being generated. However, near a refractive boundary (e.g. a nucleus within the cytoplasm), the difference between the ordinary and the extraordinary beam will generate a relief in the image. Differential interference contrast requires a polarized light source to function; two polarizing filters have to be fitted in the light path, one below the condenser (the polarizer), and the other above the objective (the analyzer).

Another microscopic technique using interference is interference reflection microscopy (also known as reflected interference contrast, or RIC). It is used to examine the adhesion of cells to a glass surface, using polarized light of a narrow range of wavelengths to be reflected whenever there is an interface between two substances with different refractive indices. Whenever a cell is attached to the glass surface, reflected light from the glass and that from the attached cell will interfere. If there is no cell attached to the glass, there will be no interference.

A fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” refers to any microscope that uses fluorescence to generate an image, whether it is a more simple set up like an epifluorescence microscope, or a more complicated design such as a confocal microscope, which uses optical sectioning to get better resolution of the fluorescent image.

Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. COLM provides an alternative microscopy for fast 3D imaging of large clarified samples. COLM interrogates large immunostained tissues, permits increased speed of acquisition and results in a higher quality of generated data.

In single plane illumination microscopy (SPIM), also known as light sheet microscopy, only the fluorophores in the focal plane of the detection objective lens are illuminated. The light sheet is a beam that is collimated in one and focused in the other direction. Since no fluorophores are excited outside the detectors' focal plane, the method also provides intrinsic optical sectioning. Moreover, when compared to conventional microscopy, light sheet methods exhibit reduced photobleaching and lower phototoxicity, and often enable far more scans per specimen. By rotating the specimen, the technique can image virtually any plane with multiple views obtained from different angles. For every angle, however, only a relatively shallow section of the specimen is imaged with high resolution, whereas deeper regions appear increasingly blurred.

Super-resolution microscopy is a form of light microscopy. Due to the diffraction of light, the resolution of conventional light microscopy is limited as stated by Ernst Abbe in 1873. A good approximation of the resolution attainable is the FWHM (full width at half-maximum) of the point spread function, and a precise widefield microscope with high numerical aperture and visible light usually reaches a resolution of ˜250 nm. Super-resolution techniques allow the capture of images with a higher resolution than the diffraction limit. They fall into two broad categories, “true” super-resolution techniques, which capture information contained in evanescent waves, and “functional” super-resolution techniques, which use experimental techniques and known limitations on the matter being imaged to reconstruct a super-resolution image.

Laser microscopy uses laser illumination sources in various forms of microscopy. For instance, laser microscopy focused on biological applications uses ultrashort pulse lasers, or femtosecond lasers, in a number of techniques including nonlinear microscopy, saturation microscopy, and multiphoton fluorescence microscopy such as two-photon excitation microscopy (a fluorescence imaging technique that allows imaging of living tissue up to a very high depth, e.g. one millimeter)

In electron microscopy (EM), a beam of electrons is used to illuminate a specimen and produce a magnified image. An electron microscope has greater resolving power than a light-powered optical microscope because electrons have wavelengths about 100,000 times shorter than visible light (photons). They can achieve better than 50 pm resolution and magnifications of up to about 10,000,000× whereas ordinary, non-confocal light microscopes are limited by diffraction to about 200 nm resolution and useful magnifications below 2000×. The electron microscope uses electrostatic and electromagnetic “lenses” to control the electron beam and focus it to form an image. These lenses are analogous to but different from the glass lenses of an optical microscope that form a magnified image by focusing light on or through the specimen. Electron microscopes are used to observe a wide range of biological and inorganic specimens including microorganisms, cells, large molecules, biopsy samples, metals, and crystals. Industrially, the electron microscope is often used for quality control and failure analysis. Examples of electron microscopy include Transmission electron microscopy (TEM), Scanning electron microscopy (SEM), reflection electron microscopy (REM), Scanning transmission electron microscopy (STEM) and low-voltage electron microscopy (LVEM).

Scanning probe microscopy (SPM) is a branch of microscopy that forms images of surfaces using a physical probe that scans the specimen. An image of the surface is obtained by mechanically moving the probe in a raster scan of the specimen, line by line, and recording the probe-surface interaction as a function of position. Examples of SPM include atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM).

Intact tissue expansion microscopy (exM) enables imaging of thick preserve specimens with roughly 70 nm lateral resolution. Using ExM the optical diffraction limit is circumvented by physically expanding a biological specimen before imaging, thus bringing sub-diffraction limited structures into the size range viewable by a conventional diffraction-limited microscope. ExM can image biological specimens at the voxel rates of a diffraction limited microscope, but with the voxel sizes of a super-resolution microscope. Expanded samples are transparent, and index-matched to water, as the expanded material is >99% water. Techniques of expansion microscopy are known in the art, e.g., as disclosed in Gao et al., Q&A: Expansion Microscopy, BMC Biol. 2017; 15:50.

### Screening Methods

The methods disclosed herein also provide for a method of screening a candidate agent to determine whether the candidate agent modulates gene expression of a nucleic acid in a cell in an intact tissue. The method comprises performing the steps disclosed herein to determine the gene sequence of a target nucleic acid in the cell in an intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In some aspects, the detecting includes performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In certain aspects, the flow cytometry is mass cytometry or fluorescence-activated flow cytometry. In some other aspects, the detecting includes performing microscopy, scanning mass spectrometry or other imaging techniques described herein. In such aspects, the detecting includes determining a signal, e.g., a fluorescent signal.

By “test agent,” “candidate agent,” and grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in a subject assay.

A variety of different candidate agents may be screened by the above methods. Candidate agents encompass numerous chemical classes, e.g., small organic compounds having a molecular weight of more than 50 daltons (e.g., at least about 50 Da, at least about 100 Da, at least about 150 Da, at least about 200 Da, at least about 250 Da, or at least about 500 Da) and less than about 20,000 daltons, less than about 10,000 daltons, less than about 5,000 daltons, or less than about 2,500 daltons. For example, in some embodiments, a suitable candidate agent is an organic compound having a molecular weight in a range of from about 500 Da to about 20,000 Da, e.g., from about 500 Da to about 1000 Da, from about 1000 Da to about 2000 Da, from about 2000 Da to about 2500 Da, from about 2500 Da to about 5000 Da, from about 5000 Da to about 10,000 Da, or from about 10,000 Da to about 20,000 Da.

Candidate agents can include functional groups necessary for structural interaction with proteins, e.g., hydrogen bonding, and can include at least an amine, carbonyl, hydroxyl or carboxyl group, or at least two of the functional chemical groups. The candidate agents can include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, candidate modulators are synthetic compounds. Any number of techniques is available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the candidate agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate agents include proteins (including antibodies, antibody fragments (i.e., a fragment containing an antigen-binding region, single chain antibodies, and the like), nucleic acids, and chemical moieties. In one embodiment, the candidate agents are naturally occurring proteins or fragments of naturally occurring proteins. Thus, for example, cellular extracts containing proteins, or random or directed digests of proteinaceous cellular extracts, may be tested. In this way libraries of prokaryotic and eukaryotic proteins may be made for screening. Other embodiments include libraries of bacterial, fungal, viral, and mammalian proteins (e.g., human proteins).

In one embodiment, the candidate agents are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

### Devices and Systems

Also included are devices for performing aspects of the subject methods. The subject devices may include, for example, imaging chambers, electrophoresis apparatus, flow chambers, microscopes, needles, tubing, pumps.

The present disclosure also provides systems for performing the subject methods. Systems may include, e.g. a power supply, a refrigeration unit, waste, a heating unit, a pump, etc. Systems may also include any of the reagents described herein, e.g. imaging buffer, wash buffer, strip buffer, Nissl and DAPI solutions. Systems in accordance with certain embodiments may also include a microscope and/or related imaging equipment, e.g., camera components, digital imaging components and/or image capturing equipment, computer processors configured to collect images according to one or more user inputs, and the like.

As discussed above, the systems described herein include a fluidics device having an imaging chamber and a pump; and a processor unit configured to perform the methods for in situ gene sequencing of a target nucleic acid in a cell in an intact tissue described herein. In some embodiments, the system enables the automation of the disclosed methods, including, but not limited to, repeated rounds of hybridization of probes with DNA embedded in a gel, ligation of fluorescently labeled oligonucleotides onto these probes, washing off the excess probes, imaging, and stripping off the probes for the next round of sequencing. In some embodiments, the system may allow for continual operation. In some embodiments, the system includes an imaging chamber for flowing sequencing chemicals involved in in situ DNA sequencing over a sample. In some embodiments, the system of fluidics and pumps control sequencing chemical delivery to the sample.

Buffers may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. For example, a first tube having a first and second end may be attached to a first port and a second tube having a first and second end may be attached to a second port, where the first end of the first tube is attached to the first port and the second end of the first tube is operably linked to a receptacle, e.g. a cooling unit, heating unit, filtration unit, waste receptacle, etc.; and the first end of the second tube is attached to the second port and the second end of the second tube is operably linked to a receptacle, e.g. a cooling unit, beaker on ice, filtration unit, waste receptacle, etc.

In some embodiments, the system includes a non-transitory computer-readable storage medium that has instructions, which when executed by the processor unit, cause the processor unit to control the delivery of chemicals and synchronize this process with a microscope. In some embodiments, the non-transitory computer-readable storage medium includes instructions, which when executed by the processor unit, cause the processor unit to measure an optical signal.

### Utility

The devices, methods, and systems herein find a number of uses in the art such as in biomedical research and/or clinical diagnostics. For example, in biomedical research, applications include, but are not limited to, spatially resolved gene expression analysis for fundamental biology or drug screening. In clinical diagnostics, applications include, but are not limited to, detecting gene markers such as disease, immune responses, bacterial or viral DNA/RNA for patient samples. Examples of advantages of the methods described herein include efficiency, where it takes merely 3 or 4 days to obtain final data from a raw sample, providing speeds much faster than existing microarray or sequencing technology; highly multiplexed (up to 1000 genes); single-cell and single-molecule sensitivity; preserved tissue morphology; and/or high signal-to-noise ratio with low error rates.

The subject methods provide multi-feature integration with next-generation 3D in situ sequencing by combining endogenous transcript detection via in situ sequencing with expressed exogenous barcode detection. Barcoded viruses are used to convert anatomical information signals into in situ sequencing compatible signals. For example, barcoded viruses can be used during electrophysiology recording to barcode individual cells for assignment of in situ sequencing data to electrophysiology data and cell morphologies.

In certain aspects, the subject methods may be applied to the study of molecular-defined cell types and activity-regulated gene expression in the visual cortex, and to be scalable to larger 3D tissue blocks to visualize short- and long-range spatial organization of cortical neurons on a volumetric scale not previously accessible. In some embodiments, the methods disclosed herein may be adapted to image DNA-conjugated antibodies for highly multiplexed protein detection.

The devices, methods, and systems of the invention can also be generalized to study a number of heterogeneous cell populations in diverse tissues. Without being bound by any scientific theory, the brain poses special challenges well suited to the methods disclosed herein. For example, the polymorphic activity-regulated gene (ARG) expression observed across different cell types is likely to depend on both intrinsic cell-biological properties (such as signal transduction pathway-component expression), and on extrinsic properties such as neural circuit anatomy that routes external sensory information to different cells (here in visual cortex). In such cases, in situ transcriptomics can effectively link imaging-based molecular information with anatomical and activity information, thus elucidating brain function and dysfunction.

The devices, methods, and systems disclosed herein enable cellular components, e.g. lipids that normally provide structural support but that hinder visualization of subcellular proteins and molecules to be removed while preserving the 3-dimensional architecture of the cells and tissue because the sample is crosslinked to a hydrogel that physically supports the ultrastructure of the tissue. This removal renders the interior of biological specimen substantially permeable to light and/or macromolecules, allowing the interior of the specimen, e.g. cells and subcellular structures, to be microscopically visualized without time-consuming and disruptive sectioning of the tissue. The procedure is also more rapid than procedures commonly used in the art, as clearance and permeabilization, typically performed in separate steps, may be combined in a single step of removing cellular components. Additionally, the specimen can be iteratively stained, unstained, and re-stained with other reagents for comprehensive analysis. Further functionalization with the polymerizable acrylamide moiety enables amplicons to be covalently anchored within the polyacrylamide network at multiple sites.

In one example, the subject devices, methods, and systems may be employed to evaluate, diagnose or monitor a disease. “Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), prediction of a subject's responsiveness to treatment for a disease or disorder (e.g., a positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy). For example, a biopsy may be prepared from a cancerous tissue and microscopically analyzed to determine the type of cancer, the extent to which the cancer has developed, whether the cancer will be responsive to therapeutic intervention, etc.

The subject devices, methods, and systems also provide a useful technique for screening candidate therapeutic agents for their effect on a tissue or a disease. For example, a subject, e.g. a mouse, rat, dog, primate, human, etc. may be contacted with a candidate agent, an organ or a biopsy thereof may be prepared by the subject methods, and the prepared specimen microscopically analyzed for one or more cellular or tissue parameters. Parameters are quantifiable components of cells or tissues, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may include detecting cellular viability, tissue vascularization, the presence of immune cell infiltrates, efficacy in altering the progression of the disease, etc. In some embodiments, the screen includes comparing the analyzed parameter(s) to those from a control, or reference, sample, e.g., a specimen similarly prepared from a subject not contacted with the candidate agent. Candidate agents of interest for screening include known and unknown compounds that encompass numerous chemical classes, primarily organic molecules, which may include organometallic molecules, inorganic molecules, genetic sequences, etc. Candidate agents of interest for screening also include nucleic acids, for example, nucleic acids that encode siRNA, shRNA, antisense molecules, or miRNA, or nucleic acids that encode polypeptides. An important aspect of the invention is to evaluate candidate drugs, including toxicity testing; and the like. Evaluations of tissue samples using the subject methods may include, e.g., genetic, transcriptomic, genomic, proteomic, and/or metabolomics analyses.

The subject devices, methods, and systems may also be used to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, ancestry, and the like. Such detection may be used in, for example, diagnosing and monitoring disease as, e.g., described above, in personalized medicine, and in studying paternity.

A database of analytic information can be compiled. These databases may include results from known cell types, references from the analysis of cells treated under particular conditions, and the like. A data matrix may be generated, where each point of the data matrix corresponds to a readout from a cell, where data for each cell may include readouts from multiple labels. The readout may be a mean, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-95 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of in situ sequencing of a target nucleic acid in a cell in an intact tissue in combination with cell barcoding, the method comprising:
   introducing into the cell in the intact tissue a nucleic acid comprising a sequence encoding a messenger RNA (mRNA) transcript comprising a 3′-untranslated region (3′-UTR) comprising a cell barcode and a poly-adenylation site, wherein the cell barcode is adjacent to the poly-adenylation site, wherein the cell expresses the mRNA transcript;
   measuring morphological or functional characteristics of the cell in the intact tissue;
   sequencing the barcode of the mRNA transcript; and
   performing in situ gene sequencing of the target nucleic acid in the cell in the intact tissue, wherein the cell barcode is used for assignment of in situ sequencing data to the measured morphological or functional characteristics of the cell.
2. The method of aspect 1, wherein said measuring morphological or functional characteristics comprises performing gene expression profiling, microscopy, calcium imaging, an electrophysiology measurement, functional neuroimaging, a migration assay, an axonal growth and pathfinding assay, a phagocytosis assay, an enzymatic assay, a cell receptor assay, an ion channel assay, a signal transduction assay, or a cell secretion assay.
3. The method of aspect 2, wherein the microscopy is confocal microscopy, atomic force microscopy, super-resolution microscopy, light-sheet microscopy, two-photon microscopy, or fluorescence microscopy.
4. The method of aspect 2, wherein the electrophysiology measurement is patch clamping, electroencephalography (EEG), or magnetoencephalography (MEG).
5. The method of aspect 2, wherein the functional neuroimaging is functional magnetic resonance imaging (fMRI), positron emission tomography (PET), functional near-infrared spectroscopy (fNIRS), single-photon emission computed tomography (SPECT), or functional ultrasound imaging (fUS).
6. The method of any one of aspects 1-5, wherein the nucleic acid is introduced into the cell in vivo, ex vivo, or in vitro prior to said measuring the morphological or functional characteristics of the cell.
7. The method of any one of aspects 1-6, wherein the morphological or functional characteristics are measured in tissue of a live subject in vivo or in culture in vitro.
8. The method of any one of aspects 1-7, wherein the subject is a nonhuman animal.
9. The method of any one of aspects 7 or 8, further comprising removing the intact tissue from the subject prior to performing in situ gene sequencing.
10. The method of any one of aspects 1-9, wherein the intact tissue is a biopsy or surgical specimen.
11. The method of any one of aspects 1-10, wherein the nucleic acid encoding the mRNA transcript is introduced into the cell with a viral vector.
12. The method of any one of aspects 1-10, wherein the viral vector is an adeno-associated virus (rAAV) vector.
13. The method of any one of aspects 1-11, wherein the mRNA transcript further comprises a coding sequence encoding a protein.
14. The method of aspect 13, wherein the protein is a fluorescent protein or a bioluminescent protein.
15. The method of aspect 14, further comprising imaging the fluorescent protein or the bioluminescent protein, wherein a location of the cell expressing the fluorescent protein or the bioluminescent protein is determined from the imaging.
16. The method of aspect 15, further comprising mapping the location of the cell expressing the fluorescent protein or the bioluminescent protein onto a reference image of the intact tissue.
17. The method of aspect 15 or 16, further comprising mapping the in situ sequencing data onto the reference image of the intact tissue.
18. The method of any one of aspects 1-17, wherein the cell is a neuron.
19. The method of aspect 18, wherein the neuron is a projection neuron.
20 The method of aspect 19, wherein the viral vector is introduced into a projection of the projection neuron, wherein retrograde transport of the viral vector delivers the viral vector to the cell body of the projection neuron.
21. The method of any one of aspects 1-20, wherein said introducing the viral vector into the cell comprises stereotactic injection of the viral vector.
22. The method of any one of aspects 1-21, further comprising optogenetically modifying one or more cells in the intact tissue.
23. The method of any one of aspects 18-22, further comprising mapping functional neuroimaging data onto the reference image of the intact tissue.
24. The method of any one of aspects 1-23, further comprising fixing and permeabilizing the intact tissue.
25. The method of any one of aspects 1-24, wherein said sequencing the barcode of the mRNA transcript comprises performing single-cell 3′-RNA sequencing of the mRNA transcript.
26. The method of aspect 24, wherein said performing in situ gene sequencing comprises:
   (a) contacting the fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid;
   (b) adding ligase to ligate the second oligonucleotide and generate a closed nucleic acid circle;
   (c) performing rolling circle amplification in the presence of a nucleic acid molecule, wherein the performing comprises using the second oligonucleotide as a template and the first oligonucleotide as a primer for a polymerase to form one or more amplicons;
   (d) embedding the one or more amplicons in the presence of hydrogel subunits to form one or more hydrogel-embedded amplicons;
   (e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon;
   (f) reiterating step (e); and
   (g) imaging the one or more hydrogel-embedded amplicons to determine in situ a gene sequence of the target nucleic acid in the cell in the intact tissue.
27. The method of aspect 26, wherein the target nucleic acid is the mRNA transcript comprising the 3′-untranslated region (3′-UTR) comprising the cell barcode and the poly-adenylation site, wherein said imaging is used to determine the sequence of the cell barcode.
28 The method of aspect 26 or 27, wherein the length of the cell barcode sequence is sufficient to allow at least one pair of oligonucleotide primers to bind to the cell barcode sequence, wherein the first complementarity region of the first oligonucleotide is complementary to a first portion of the barcode sequence, wherein the second complementary region of the second oligonucleotide is complementary to a second portion of the barcode sequence, wherein the first portion of the barcode sequence is adjacent to the second portion of the barcode sequence.
29. The method of aspect 28, wherein the length of the cell barcode sequence is sufficient to allow at least two pairs of oligonucleotide primers to bind to the cell barcode sequence.
30. The method of aspect 29, wherein the length of the cell barcode sequence is sufficient to allow at least four pairs of oligonucleotide primers to bind to the cell barcode sequence.
31. The method of aspect 28, wherein the cell barcode sequence has a length of at least 40 nucleotides.
32. The method of any one of aspects 26-31, further comprising contacting the fixed and permeabilized intact tissue with a gel adaptor oligonucleotide that binds to the first oligonucleotide, wherein the gel adaptor oligonucleotide comprises a nucleotide modification at the 5′ end that links the gel adapter to the hydrogel during gelation.
33. The method of aspect 32, wherein the modification comprises an acrydite group.
34. The method of aspect 32 or 33, wherein the first oligonucleotide further comprises a common binding site for the gel adaptor oligonucleotide.
35. The method of aspect 34, wherein the common binding site for the gel adaptor oligonucleotide is adjacent to the first complementarity region of the first oligonucleotide.
36. The method of any one of aspects 32-35, further comprising barcoding a cell by contacting the cell with:
   a first probe comprising a 5′-amine modification or a 5′-biotin modification, a common gel adaptor complementary sequence that hybridizes with the gel adaptor oligonucleotide, and a unique barcode sequence; and
   a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe.
37. The method of aspect 36, wherein the second probe is a padlock probe.
38. The method of aspect 36 or 37, wherein a plurality of first probes and second probes are used to barcode a plurality of cells in the intact tissue, wherein each first probe has a different unique barcode sequence.
39. The method of any one of aspects 1-38, wherein sequencing is performed with sequential or combinatorial encoding.
40. The method of any one of aspects 1-39, further comprising preincubating the tissue sample with the polymerase for a sufficient time to allow uniform diffusion of the polymerase throughout the tissue before performing the rolling circle amplification.
41. The method of any one of aspects 1-40, wherein said imaging is performed in presence of an anti-fade buffer comprising an antioxidant.
42. The method of any one of aspects 1-41, wherein the signal is a fluorescent signal.
43 The method of aspect 42, further comprising removing the signal after imaging by contacting the hydrogel with formamide.
44. The method of aspect 42, wherein the fourth oligonucleotide is covalently linked to a fluorophore by a disulfide bond.
45. The method of aspect 44, further comprising contacting the hydrogel with a reducing agent after said imaging, wherein reduction of the disulfide bond results in cleavage of the fluorophore from the fourth oligonucleotide.
46. The method of any one of aspects 1-45, wherein the set of primers are denatured by heating before contacting the sample.
47. The method of any one of aspects 1-46, wherein the cell is present in a population of cells.
48. The method of aspect 47, wherein the population of cells comprises a plurality of cell types.
49. The method of any one of aspects 1-48, wherein the contacting the fixed and permeabilized intact tissue comprises hybridizing the primers to the same target nucleic acid.
50. The method of any one of aspects 1-49, wherein the target nucleic acid is RNA or DNA.
51. The method of aspect 50, wherein the RNA is mRNA.
52. The method of any one of aspects 1-51, wherein the second oligonucleotide comprises a padlock probe.
53. The method of any one of aspects 1-52, wherein the first complementarity region of the first oligonucleotide has a length of 19-25 nucleotides.
54. The method of any one of aspects 1-53, wherein the second complementarity region of the first oligonucleotide has a length of 6 nucleotides.
55. The method of any one of aspects 1-54, wherein the third complementarity region of the first oligonucleotide has a length of 6 nucleotides.
56. The method of any one of aspects 1-55, wherein the first complementarity region of the second oligonucleotide has a length of 6 nucleotides.
57. The method of any one of aspects 1-56, wherein the second complementarity region of the second oligonucleotide has a length of 19-25 nucleotides.
58. The method of any one of aspects 1-57, wherein the third complementarity region of the second oligonucleotide has a length of 6 nucleotides.
59. The method of any one of aspects 1-58, wherein the first complementarity region of the second oligonucleotide comprises the 5′ end of the second oligonucleotide.
60. The method of any one of aspects 1-59, wherein the third complementarity region of the second oligonucleotide comprises the 3′ end of the second oligonucleotide.
61. The method of any one of aspects 1-60, wherein the first complementarity region of the second oligonucleotide is adjacent to the third complementarity region of the second oligonucleotide.
62. The method of any one of aspects 1-61, wherein the barcode sequence of the second oligonucleotide provides barcoding information for identification of the target nucleic acid.
63. The method of any one of aspects 1-62, wherein the contacting the fixed and permeabilized intact tissue comprises hybridizing a plurality of oligonucleotide primers having specificity for different target nucleic acids.
64. The method of any one of aspects 1-63, wherein the second oligonucleotide is provided as a closed nucleic acid circle, and the step of adding ligase is omitted.
65. The method of any of aspects 1-64, wherein the melting temperature (Tₘ) of oligonucleotides is selected to minimize ligation in solution.
66. The method of any one of aspects 1-65, wherein the adding ligase comprises adding a DNA ligase.
67. The method of any one of aspects 1-66, wherein the nucleic acid molecule comprises an amine-modified nucleotide.
68. The method of aspect 67, wherein the amine-modified nucleotide comprises an acrylic acid N-hydroxysuccinimide moiety modification.
69. The method of any one of aspects 1-68, wherein the embedding comprises copolymerizing the one or more amplicons with acrylamide.
70. The method of any one of aspects 1-69, wherein the embedding comprises clearing the one or more hydrogel-embedded amplicons wherein the target nucleic acid is substantially retained in the one or more hydrogel-embedded amplicons.
71. The method of aspect 70, wherein the clearing comprises substantially removing a plurality of cellular components from the one or more hydrogel-embedded amplicons.
72. The method of aspect 70 or 71, wherein the clearing comprises substantially removing lipids or proteins, or a combination thereof from the one or more hydrogel-embedded amplicons.
73. The method of any one of aspects 1-72, wherein the contacting the one or more hydrogel-embedded amplicons comprises eliminating error accumulation as sequencing proceeds.
74. The method of any one of aspects 1-73, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).
75. The method of any one of aspects 1-74, wherein the intact tissue is a thin slice.
76. The method of aspect 75, wherein the intact tissue has a thickness of 5-20 μm.
77. The method of aspect 75 or 76, wherein the contacting the one or more hydrogel-embedded amplicons occurs four times or more.
78. The method of any one of aspects 1-77, wherein the intact tissue is a thick slice.
79 The method of aspect 78, wherein the intact tissue has a thickness of 50-200 μm.
80. The method of aspect 78 or 79, wherein the contacting the one or more hydrogel-embedded amplicons occurs six times or more.
81. The method of any one of aspects 1-80, wherein the target nucleic acid is exogenous.
82. The method of aspect 81, wherein the target nucleic acid is introduced into the cell by a viral vector.
83. The method of aspect 81 or 82, wherein the target nucleic acid is viral RNA or viral DNA.
84. The method of any one of aspects 81-83, wherein the target nucleic acid is integrated into the host genome and expressed by an endogenous promoter.
85. The method of aspect 81, wherein the target nucleic acid is introduced into the cell using a non-viral vector.
86. The method of aspect 81, wherein the target nucleic acid is introduced into the cell using a lipid nanoparticle.
87. A method of screening a candidate agent to determine whether the candidate agent modulates gene expression of a nucleic acid in a cell in an intact tissue, the method comprising performing the method of any one of aspects 1-86 to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.
88. The method of aspect 87, wherein the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy.
89. The method of aspect 87, wherein the flow cytometry is mass cytometry or fluorescence-activated flow cytometry.
90. The method of any one of aspects 87-89, wherein the detecting comprises performing microscopy, scanning mass spectrometry, or other imaging techniques 91. The method of any one of aspects 87-90, wherein the detecting comprises detecting a signal.
92. The method of aspect 87, wherein the signal is a fluorescent signal.
93. A system, comprising:
   a fluidics device, and
   a processor unit configured to perform the method of any one of aspects 1-92.
94. The system of aspect 93, further comprising an imaging chamber.
95. The system of aspect 93 or 94, further comprising a pump.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Multiple Feature Integration with Next-Generation 3D In Situ Sequencing

### Overview

Conversion of distinct types of signals of interest into barcode encoding space AAV encoding barcoded transcripts

Use of AAVretro barcoded viruses to convert anatomical information signals into in situ sequencing compatible signals

Use of cell-filling oligos during electrophysiology recording to barcode individual cells for assignment of in situ sequencing data to electrophysiology data and cell morphologies

Integration of distinct target modalities with in situ sequencing

Combining endogenous transcript detection via in situ sequencing with expressed exogenous barcode detection (ex: AAVretro barcodes) via in situ sequencing

Combining transcript detection with in situ sequencing with cell-filling cell-specific injected barcode read out with in situ sequencing

Combining transcript detection with in situ sequencing with additional directly labeled fluorescent channels, before or after sequencing has occurred

Combining transcript or other exogenous barcode detection via ex vivo in situ sequencing with in vivo information channels via computational image alignment

### Technical Description

### Retrograde Virus Barcoding

AAVs were generated in which the mRNA transcript generated upon infection of a cell with the AAV contained a barcode sequence that could specifically be read out either by 3′ single-cell RNA sequencing or by STARmap2. These transcripts contained unique sequence in their 3′ UTR adjacent to the poly-adenylation site, so that NGS reads from a 3′ polyA would read through the barcode. The barcode lengths used in these AAV are appropriate for at least one standard SNAIL probe pairs to bind, and as many as 4. Eight different uniquely barcoded AAVretro-serotype AAVs were generated (that also expressed the protein H2B-3xMyc-epitope), and eight other AAVretro, with the same barcode sequences, were generated that express the protein mScarlet (for direct fluorescent visualization). These viruses were injected into projection targets of the area of interest (0.5 μl to 1 μl stereotactic injections), such that they would retrogradely traffic and infect the cell bodies of projection neurons in the area of interest. By using STARmap2 to read out the barcodes contained in these virus, multiplex detection of projection information could be layered on top of data for other transcripts targeted by STARmap.

### In Vivo to Ex Vivo Alignment

### Experimental Procedures

Mice were made to express GCaMP6s or 6f virally (through stereotactic injection) or via transgenics (line Ai148 coupled with viral introduction of Cre into the area of interest). Additionally, a photoactivatable H2B-RFP was virally expressed through the hSyn promoter in neurons, such that UV photoactivation of the imaged region resulted in red fluorescent fiducial signals in the region of interest. Live signals (GCaMP activity) were imaged in awake mice on a two-photon microscope through a cannula window implanted into the brain. Following imaging of GCaMP activity, the imaged region was illuminated with UV light to photoactivate the nuclear RFP signals, and a structural stack at 3 μm z resolution with isosbestic GCaMP wavelength was obtained for downstream alignment. Additionally, a structural stack at the same position and z size was obtained for RFP signals. Mice were subsequently euthanized, perfused with ice cold PBS and subsequently with ice cold 4% PFA and brains were partially dissected from the head, leaving the top of the skull and headbar intact, before post-fixing overnight in 4% PFA at 4° C. Following equilibration with ice cold PBS, brains were carefully dissected from the bottom, leaving only the top skull intact, the canula still implanted into the brain tissue and the headbar still attached. Headbars/brains were placed into a custom headbar holder with a sliding platform beneath, so that a vibratome platform with gel glue could be position directly under the brain at an angle that matched the imaging angle during live imaging. The platform was raised and glue was allowed to set before lowering the platform and separating the brain from the skull and headbar (leaving just the brain embedded onto the vibratome platform, such that the horizontal sectioning plane matched the imaging plane). Sections were cut at 150 μm on the vibratome until the hole from the canula implant had just disappeared. The subsequent two sections were collected into 70% EtOH and processed for thick section STARmap as described above. During STARmap sequencing but prior to the addition of sequencing signals, the STARmap sample was imaged for RFP signal (small residual nuclear RFP signal remains) before photobleaching of any remaining signal and subsequent STARmap sequencing chemistry.

Computational procedures

A custom computational alignment pipeline was used to preprocess signals from in vivo imaging data and ex vivo STARmap nuclear RFP reference data. To enable direct, automated image alignment in 3D between STARmap RFP reference data and 2P RFP data, both datasets were passed through a pixel classifier trained specifically for either 2P or STARmap data acquisition formats, which converted raw signals into pixel probability maps for nuclei signals. The resulting images were subsequently scaled, rotated, and cropped according to the known differences in the optical set ups (pixel sizes, camera orientations), with manual fine tuning in the XY plane for rotation and cropping. Datasets were then inspected side-by-side in a custom software interface for matching cell nuclei arrangement features, which were manually selected and used to generate an initial affine transformation. Subsequently, automatic 3D affine and b-spline registration steps were performed with a custom parameter set in Elastix to map the STARmap dataset onto the in vivo reference image space, and this alignment was scored for overlap by comparing pixel covariance at a scale finer than the smallest degree of freedom allowed to the automatic registration procedure (avoiding overfitting). Areas of the imaging passing this alignment quality control scoring procedure were preserved, and other areas not passing were zeroed out. The in vivo RFP dataset was then aligned with Elastix to the GCaMP structural image, and this was further aligned with FFT-based cross-correlation fitting to the average image Z planes from activity imaging. By chaining these registrations together, the 3D footprints of segmented STARmap cells were mapped into the in vivo GCaMP imaging space, allowing for direct matching between extracted GCaMP sources and STARmap cells.

### STARpatch

### Cell-Barcoding

Per-cell barcodes were designed in two components. First, a 5′ splint sequence, containing either a 5′ amine modification (for fixation or subsequent modification) or a 5′ biotin modification (for facilitated polar trafficking through cells), a common gel adaptor complementary sequence, and a unique 40 nucleotide (nt) sequence. Second, a padlock probe containing 20 nt of sequence at each end complementary to each half of the unique 40 nt sequence of the first probe, flanking a sequencing encoding sequence (for example, the sequential encoding sequence for a particular round and base). The pair of components, pre-hybridized together, constituted a barcode that could follow through the STARmap2 procedures of fixation, hybridization (with the gel adaptor oligo), polymerization into the hydrogel, ligation, and amplification by RCA, enabling STARmap2 read out of the encoded sequence along with any endogenous signals being detected. See below for methodological description of an example use of these cell barcodes for cell tagging and morphological reconstruction following patch clamp recording in an intact tissue volume.

Distinct cell barcodes were chosen and recorded, and included in the internal solution for a given whole cell patch performed in tissue slice (see Electrophysiology below). Following completion of electrophysiology, tissue was immediately fixed in 4% PFA overnight at 4° C., washed in ice cold 1X PBS, and preserved in 70% EtOH before proceeding into the thick section sample prep and library generation procedure as described above. Cell barcodes were read out with sequential SCAL following sequencing cycles for endogenous RNA targets and other barcodes (in this case, the AAVretro barcodes), allowing the cell-barcode associated with each whole-cell patch electrophysiology dataset to be tied to a particular cell identified by its corresponding barcode expression in STARmap2.

### Electrophysiology

Acute coronal brain slices for patch clamp experiments were prepared from mice that had been previously injected with barcoded AAVretro (mScarlet) as described above. Acute slices were prepared as previously described. Briefly, animals were anesthetized with isoflurane until absence of toe and tail reflex, then trans-cardially perfused with ice-cold, carbogen-bubbled (95% O2, 5% CO₂) NMDG artificial cerebrospinal fluid (aCSF; NMDF 92 mM, KCl 2.5 mM, NaH₂PO₄ 1.25 mM, NaHCO₃30 mM, HEPES 20 mM, glucose 25 mM, thiourea 2 mM, Na-ascorbate 5 mM, Na-pyruvate 3 mM, CaCl₂) 0.5 mM, MgSO₄ 10 mM. pH 7.3-7.4 with HCl, osmolarity 300-310). The subject was then decapitated, the brain carefully dissected out of the skull, blocked, affixed to a vibratome stage, and submerged in carbogen-bubbled NMDG aCSF. 150 μm horizontal sections were cut using a Leica vibratome, then transferred to 34 C NMDG aCSF for 10-12 minutes, before being transferred to RT HEPES holding aCSF (NaCl 92 mM, KCl 2.5 mM, NaH₂PO₄ 1.25 mM, NaHCO₃30 mM, HEPES 20 mM, glucose 25 mM, thiourea 2 mM, Na-ascorbate 5 mM, Na-pyruvate 3 mM, CaCl₂) 2 mM, MgSO₄, 2 mM. pH 7.3-7.4 with HCl or NaOH, as needed, osmolarity 300-310 mOsm). Slices were kept in this solution for a minimum of 1 hour (hr) and until use.

For electrophysiological recordings, slices were transferred to an immersion stage and continuously perfused with carbogenated, RT aCSF (NaCl 119 mM, KCl 2.5 mM, NaH₂PO₄ 1.25 mM, NaHCO₃24 mM, glucose 12.5 mM, CaCl₂) 2 mM, MgSO₄ 2 mM. pH 7.3-7.4 with NaOH or HCl as needed, osmolarity 300-310 mOsm). Appropriate horizontal sections were visually identified by typical anatomic landmarks and layer 5 of the orbitofrontal cortex was identified under low magnification by typical appearance on a Leica DM-LFSA microscope. 5-8 MQ resistance patch pipettes pulled with a P-97 micropipette puller from 1-mm micro-haematocrit-tubes were filled with internal solution (stock internal solution was made at 1.1× concentration to allow dilution with DNA oligonucleotides. Final concentrations are KGluconate 145 mM, HEPES 10 mM, EGTA 1 mM, MgCl₂ 2 mM, ATP 2 mM and DNA oligonucleotides as noted in text, pH 7.3 with KOH, osmolarity 290-300 mOsm). In some cases, 5 mM biocytin was included in the internal solution. Whole cell access was obtained from individual neurons after acquisition of giga-ohm seal recording with AxoClamp 700B amplifier and digitized with DigiData 1440 using pClamp software. Access quality was assessed in voltage clamp (VC) before running a series of protocols in current clamp (CC), as per refs, including 25 pA/s current injection ramp run until action potential generation, 1s square-pulse injection from −110 pA to rheobase+160 pA with delta 20 pA, 3 ms square pulse injection from 100 pA to action potential threshold with 10 pA delta, and alternating+200 pA/−200 pA square pulse injection. Total access time was recorded for individual cells and was approximately 25 minutes. Data were then analyzed in Clampfit.

What is claimed is:

1. A method of in situ sequencing of a target nucleic acid in a cell in an intact tissue in combination with cell barcoding, the method comprising:
introducing into the cell in the intact tissue a nucleic acid comprising a sequence encoding a messenger RNA (mRNA) transcript comprising a 3′-untranslated region (3′-UTR) comprising a cell barcode and a poly-adenylation site, wherein the cell barcode is adjacent to the poly-adenylation site, wherein the cell expresses the mRNA transcript;
measuring morphological or functional characteristics of the cell in the intact tissue;
sequencing the barcode of the mRNA transcript; and
performing in situ gene sequencing of the target nucleic acid in the cell in the intact tissue, wherein the cell barcode is used for assignment of in situ sequencing data to the measured morphological or functional characteristics of the cell.

2. The method of claim 1, wherein said measuring morphological or functional characteristics comprises performing gene expression profiling, microscopy, calcium imaging, an electrophysiology measurement, functional neuroimaging, a migration assay, an axonal growth and pathfinding assay, a phagocytosis assay, an enzymatic assay, a cell receptor assay, an ion channel assay, a signal transduction assay, or a cell secretion assay.

3. The method of claim 2, wherein the microscopy is confocal microscopy, atomic force microscopy, super-resolution microscopy, light-sheet microscopy, two-photon microscopy, or fluorescence microscopy.

4. The method of claim 2, wherein the electrophysiology measurement is patch clamping, electroencephalography (EEG), or magnetoencephalography (MEG).

5. The method of claim 2, wherein the functional neuroimaging is functional magnetic resonance imaging (fMRI), positron emission tomography (PET), functional near-infrared spectroscopy (fNIRS), single-photon emission computed tomography (SPECT), or functional ultrasound imaging (fUS).

6. The method of any one of claims 1-5, wherein the nucleic acid is introduced into the cell in vivo, ex vivo, or in vitro prior to said measuring the morphological or functional characteristics of the cell.

7. The method of any one of claims 1-6, wherein the morphological or functional characteristics are measured in tissue of a live subject in vivo or in culture in vitro.

8. The method of any one of claims 1-7, wherein the subject is a nonhuman animal.

9. The method of any one of claim 7 or 8, further comprising removing the intact tissue from the subject prior to performing in situ gene sequencing.

10. The method of any one of claims 1-9, wherein the intact tissue is a biopsy or surgical specimen.

11. The method of any one of claims 1-10, wherein the nucleic acid encoding the mRNA transcript is introduced into the cell with a viral vector.

12. The method of any one of claims 1-10, wherein the viral vector is an adeno-associated virus (rAAV) vector.

13. The method of any one of claims 1-11, wherein the mRNA transcript further comprises a coding sequence encoding a protein.

14. The method of claim 13, wherein the protein is a fluorescent protein or a bioluminescent protein.

15. The method of claim 14, further comprising imaging the fluorescent protein or the bioluminescent protein, wherein a location of the cell expressing the fluorescent protein or the bioluminescent protein is determined from the imaging.

16. The method of claim 15, further comprising mapping the location of the cell expressing the fluorescent protein or the bioluminescent protein onto a reference image of the intact tissue.

17. The method of claim 15 or 16, further comprising mapping the in situ sequencing data onto the reference image of the intact tissue.

18. The method of any one of claims 1-17, wherein the cell is a neuron.

19. The method of claim 18, wherein the neuron is a projection neuron.

20. The method of claim 19, wherein the viral vector is introduced into a projection of the projection neuron, wherein retrograde transport of the viral vector delivers the viral vector to the cell body of the projection neuron.

21. The method of any one of claims 1-20, wherein said introducing the viral vector into the cell comprises stereotactic injection of the viral vector.

22. The method of any one of claims 1-21, further comprising optogenetically modifying one or more cells in the intact tissue.

23. The method of any one of claims 18-22, further comprising mapping functional neuroimaging data onto the reference image of the intact tissue.

24. The method of any one of claims 1-23, further comprising fixing and permeabilizing the intact tissue.

25. The method of any one of claims 1-24, wherein said sequencing the barcode of the mRNA transcript comprises performing single-cell 3′-RNA sequencing of the mRNA transcript.

26. The method of claim 24, wherein said performing in situ gene sequencing comprises:
(a) contacting the fixed and permeabilized intact tissue with at least a pair of oligonucleotide primers under conditions to allow for specific hybridization, wherein the pair of primers comprise a first oligonucleotide and a second oligonucleotide;
wherein each of the first oligonucleotide and the second oligonucleotide comprises a first complementarity region, a second complementarity region sequence, and a third complementarity region; wherein the second oligonucleotide further comprises a barcode sequence;
wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid;
(b) adding ligase to ligate the second oligonucleotide and generate a closed nucleic acid circle;
(c) performing rolling circle amplification in the presence of a nucleic acid molecule, wherein the performing comprises using the second oligonucleotide as a template and the first oligonucleotide as a primer for a polymerase to form one or more amplicons;
(d) embedding the one or more amplicons in the presence of hydrogel subunits to form one or more hydrogel-embedded amplicons;
(e) contacting the one or more hydrogel-embedded amplicons having the barcode sequence with a set of sequencing primers under conditions to allow for ligation, wherein the set of sequencing primers comprises a third oligonucleotide configured to decode bases and a fourth oligonucleotide configured to convert decoded bases into a signal, wherein the ligation only occurs when both the third oligonucleotide and the fourth oligonucleotide are complementary to adjacent sequences of the same amplicon;
(f) reiterating step (e); and
(g) imaging the one or more hydrogel-embedded amplicons to determine in situ a gene sequence of the target nucleic acid in the cell in the intact tissue.

27. The method of claim 26, wherein the target nucleic acid is the mRNA transcript comprising the 3′-untranslated region (3′-UTR) comprising the cell barcode and the poly-adenylation site, wherein said imaging is used to determine the sequence of the cell barcode.

28. The method of claim 26 or 27, wherein the length of the cell barcode sequence is sufficient to allow at least one pair of oligonucleotide primers to bind to the cell barcode sequence, wherein the first complementarity region of the first oligonucleotide is complementary to a first portion of the barcode sequence, wherein the second complementary region of the second oligonucleotide is complementary to a second portion of the barcode sequence, wherein the first portion of the barcode sequence is adjacent to the second portion of the barcode sequence.

29. The method of claim 28, wherein the length of the cell barcode sequence is sufficient to allow at least two pairs of oligonucleotide primers to bind to the cell barcode sequence.

30. The method of claim 29, wherein the length of the cell barcode sequence is sufficient to allow at least four pairs of oligonucleotide primers to bind to the cell barcode sequence.

31. The method of claim 28, wherein the cell barcode sequence has a length of at least 40 nucleotides.

32. The method of any one of claims 26-31, further comprising contacting the fixed and permeabilized intact tissue with a gel adaptor oligonucleotide that binds to the first oligonucleotide, wherein the gel adaptor oligonucleotide comprises a nucleotide modification at the 5′ end that links the gel adapter to the hydrogel during gelation.

33. The method of claim 32, wherein the modification comprises an acrydite group.

34. The method of claim 32 or 33, wherein the first oligonucleotide further comprises a common binding site for the gel adaptor oligonucleotide.

35. The method of claim 34, wherein the common binding site for the gel adaptor oligonucleotide is adjacent to the first complementarity region of the first oligonucleotide.

36. The method of any one of claims 32-35, further comprising barcoding a cell by contacting the cell with:
a first probe comprising a 5′-amine modification or a 5′-biotin modification, a common gel adaptor complementary sequence that hybridizes with the gel adaptor oligonucleotide, and a unique barcode sequence; and
a second probe comprising a first sequence that is complementary to a first portion of the unique barcode sequence and a second sequence that is complementary to a second portion of the unique barcode sequence, wherein the first sequence and the second sequence flank a sequencing encoding sequence, wherein hybridization of the first probe and the second probe results in formation of a barcoding complex comprising the first probe and the second probe.

37. The method of claim 36, wherein the second probe is a padlock probe.

38. The method of claim 36 or 37, wherein a plurality of first probes and second probes are used to barcode a plurality of cells in the intact tissue, wherein each first probe has a different unique barcode sequence.

39. The method of any one of claims 1-38, wherein sequencing is performed with sequential or combinatorial encoding.

40. The method of any one of claims 1-39, further comprising preincubating the tissue sample with the polymerase for a sufficient time to allow uniform diffusion of the polymerase throughout the tissue before performing the rolling circle amplification.

41. The method of any one of claims 1-40, wherein said imaging is performed in presence of an anti-fade buffer comprising an antioxidant.

42. The method of any one of claims 1-41, wherein the signal is a fluorescent signal.

43. The method of claim 42, further comprising removing the signal after imaging by contacting the hydrogel with formamide.

44. The method of claim 42, wherein the fourth oligonucleotide is covalently linked to a fluorophore by a disulfide bond.

45. The method of claim 44, further comprising contacting the hydrogel with a reducing agent after said imaging, wherein reduction of the disulfide bond results in cleavage of the fluorophore from the fourth oligonucleotide.

46. The method of any one of claims 1-45, wherein the set of primers are denatured by heating before contacting the sample.

47. The method of any one of claims 1-46, wherein the cell is present in a population of cells.

48. The method of claim 47, wherein the population of cells comprises a plurality of cell types.

49. The method of any one of claims 1-48, wherein the contacting the fixed and permeabilized intact tissue comprises hybridizing the primers to the same target nucleic acid.

50. The method of any one of claims 1-49, wherein the target nucleic acid is RNA or DNA.

51. The method of claim 50, wherein the RNA is mRNA.

52. The method of any one of claims 1-51, wherein the second oligonucleotide comprises a padlock probe.

53. The method of any one of claims 1-52, wherein the first complementarity region of the first oligonucleotide has a length of 19-25 nucleotides.

54. The method of any one of claims 1-53, wherein the second complementarity region of the first oligonucleotide has a length of 6 nucleotides.

55. The method of any one of claims 1-54, wherein the third complementarity region of the first oligonucleotide has a length of 6 nucleotides.

56. The method of any one of claims 1-55, wherein the first complementarity region of the second oligonucleotide has a length of 6 nucleotides.

57. The method of any one of claims 1-56, wherein the second complementarity region of the second oligonucleotide has a length of 19-25 nucleotides.

58. The method of any one of claims 1-57, wherein the third complementarity region of the second oligonucleotide has a length of 6 nucleotides.

59. The method of any one of claims 1-58, wherein the first complementarity region of the second oligonucleotide comprises the 5′ end of the second oligonucleotide.

60. The method of any one of claims 1-59, wherein the third complementarity region of the second oligonucleotide comprises the 3′ end of the second oligonucleotide.

61. The method of any one of claims 1-60, wherein the first complementarity region of the second oligonucleotide is adjacent to the third complementarity region of the second oligonucleotide.

62. The method of any one of claims 1-61, wherein the barcode sequence of the second oligonucleotide provides barcoding information for identification of the target nucleic acid.

63. The method of any one of claims 1-62, wherein the contacting the fixed and permeabilized intact tissue comprises hybridizing a plurality of oligonucleotide primers having specificity for different target nucleic acids.

64. The method of any one of claims 1-63, wherein the second oligonucleotide is provided as a closed nucleic acid circle, and the step of adding ligase is omitted.

65. The method of any of claims 1-64, wherein the melting temperature (Tₘ) of oligonucleotides is selected to minimize ligation in solution.

66. The method of any one of claims 1-65, wherein the adding ligase comprises adding a DNA ligase.

67. The method of any one of claims 1-66, wherein the nucleic acid molecule comprises an amine-modified nucleotide.

68. The method of claim 67, wherein the amine-modified nucleotide comprises an acrylic acid N-hydroxysuccinimide moiety modification.

69. The method of any one of claims 1-68, wherein the embedding comprises copolymerizing the one or more amplicons with acrylamide.

70. The method of any one of claims 1-69, wherein the embedding comprises clearing the one or more hydrogel-embedded amplicons wherein the target nucleic acid is substantially retained in the one or more hydrogel-embedded amplicons.

71. The method of claim 70, wherein the clearing comprises substantially removing a plurality of cellular components from the one or more hydrogel-embedded amplicons.

72. The method of claim 70 or 71, wherein the clearing comprises substantially removing lipids or proteins, or a combination thereof from the one or more hydrogel-embedded amplicons.

73. The method of any one of claims 1-72, wherein the contacting the one or more hydrogel-embedded amplicons comprises eliminating error accumulation as sequencing proceeds.

74. The method of any one of claims 1-73, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

75. The method of any one of claims 1-74, wherein the intact tissue is a thin slice.

76. The method of claim 75, wherein the intact tissue has a thickness of 5-20 μm.

77. The method of claim 75 or 76, wherein the contacting the one or more hydrogel-embedded amplicons occurs four times or more.

78. The method of any one of claims 1-77, wherein the intact tissue is a thick slice.

79. The method of claim 78, wherein the intact tissue has a thickness of 50-200 μm.

80. The method of claim 78 or 79, wherein the contacting the one or more hydrogel-embedded amplicons occurs six times or more.

81. The method of any one of claims 1-80, wherein the target nucleic acid is exogenous.

82. The method of claim 81, wherein the target nucleic acid is introduced into the cell by a viral vector.

83. The method of claim 81 or 82, wherein the target nucleic acid is viral RNA or viral DNA.

84. The method of any one of claims 81-83, wherein the target nucleic acid is integrated into the host genome and expressed by an endogenous promoter.

85. The method of claim 81, wherein the target nucleic acid is introduced into the cell using a non-viral vector.

86. The method of claim 81, wherein the target nucleic acid is introduced into the cell using a lipid nanoparticle.

87. A method of screening a candidate agent to determine whether the candidate agent modulates gene expression of a nucleic acid in a cell in an intact tissue, the method comprising performing the method of any one of claims 1-86 to determine the gene sequence of the target nucleic acid in the cell in the intact tissue, and
detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

88. The method of claim 87, wherein the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy.

89. The method of claim 87, wherein the flow cytometry is mass cytometry or fluorescence-activated flow cytometry.

90. The method of any one of claims 87-89, wherein the detecting comprises performing microscopy, scanning mass spectrometry, or other imaging techniques

91. The method of any one of claims 87-90, wherein the detecting comprises detecting a signal.

92. The method of claim 87, wherein the signal is a fluorescent signal.

93. A system, comprising:
a fluidics device, and
a processor unit configured to perform the method of any one of claims 1-92.

94. The system of claim 93, further comprising an imaging chamber.

95. The system of claim 93 or 94, further comprising a pump.
